(19) United States
(12) Patent Application Publication
(10) Pub. No.: US 2007/0190019 A1
(43) Pub. Date: Aug. 16, 2007

(54) Compositions and methods for topical administration

(76) Inventors: Chunfeng Guo, Woburn, MA (US); Thomas Chan, Hopkinton, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/562,113

(22) PCT Filed: Jun. 23, 2004
(86) PCT No.: PCT/US04/20037

§ 371 Date: Mar. 27, 2007

Related U.S. Application Data
(60) Provisional application No. 60/480,230, filed on Jun. 23, 2003.

(51) Int. Cl. A61K 31/74

(52) U.S. Cl. 424/78.05

(57) ABSTRACT

Vanishing cream base compositions and vanishing cream compositions suitable for topical application of an active agent to an animal or plant comprising water, at least one alcohol, a polymeric thickening agent, a skin penetration enhancing compound, and an emulsifying agent are provided. Methods for the use and manufacture of such creams are also disclosed.

### CROSS REFERENCE TO RELATED APPLICATION

This application claims the benefit of priority from U.S. Provisional Ser. No. 60/480,230, filed Jun. 23, 2003. This application, in its entirety, is incorporated herein.

The present invention relates generally to compositions for administration of active agents through the skin and other membranes and methods of using the same and to methods for administering active agents using these compositions.

The present invention relates to compositions effective for the topical administration across skin and other membranes of active agents and to the methods for treating and/or preventing symptoms and/or disorders associated with disease or hormonal imbalances.

One embodiment of the present invention provides for a vanishing cream base composition suitable for topical application of an active agent to an animal or plant comprising:
a. from about 20 to about 70 wt. % water, based on the total weight of cream base composition;
b. at least about 10 wt. %, based on the total weight of cream base, of at least one alcohol;
c. at least one polymeric thickening agent, optionally having a nitrogen containing acrylic unit;
d. a penetration enhancing effective amount of a skin penetration enhancing compound; and
e. an emulsifying agent;
wherein said cream base is stable upon storage at ambient conditions, and clarifies at a temperature greater than 30° C.

Another embodiment of the present invention provides for a vanishing cream composition suitable for topical application of an active agent to an animal or plant comprising:
a. from about 20 to about 70 wt. % water, based on the total weight of cream base composition;
b. at least about 10 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. an active agent;
wherein said cream base is stable upon storage at ambient conditions, and clarifies at a temperature greater than 30° C.

Another embodiment of the present invention provides a vanishing cream base composition suitable for topical application of a non-ionic active agent to an animal or plant comprising:
a. from about 20 to about 50 wt. % water, based on the total weight of cream base composition;
b. at least about 30 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent;
wherein said cream base is stable upon storage at ambient conditions, and clarifies at a temperature greater than 30° C.

Another embodiment of the present invention provides a vanishing cream base composition suitable for topical application of an ionic active agent to an animal or plant comprising:
a. at least about 55 wt. % water, based on the total weight of cream base composition;
b. at least about 15 wt. %, based on the total weight of cream base, of at least one alcohol;
c. at least one polymeric thickening agent comprising a nitrogen containing acrylic unit;
d. a penetration enhancing effective amount of a skin penetration enhancing compound; and
e. an emulsifying agent;
wherein said cream base is stable upon storage at ambient conditions, and clarifies at a temperature greater than 30° C.

Another embodiment of the present invention provides a vanishing cream composition suitable for topical application to an animal or plant comprising:
a. from about 20 to about 50 wt. % water, based on the total weight of cream base composition;
b. at least about 30 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. a non-ionic active agent; and
f. an emulsifying agent;
wherein said cream base is stable upon storage at ambient conditions, and clarifies at a temperature greater than 30° C.

Another embodiment of the present invention provides for a vanishing cream composition suitable for topical application of an ionic active agent to an animal or plant comprising:
a. at least about 55 wt. % water, based on the total weight of cream base composition;
b. at least about 15 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. an ionic active agent;
wherein said cream base is stable upon storage at ambient conditions, and clarifies at a temperature greater than 30° C.

Still another embodiment of the present invention provides a method of forming a vanishing cream suitable for administering a non-ionic active agent to an animal or plant comprising, mixing:
a. from about 20 to about 50 wt. % water, based on the total weight of cream base composition;
b. at least about 30 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. a non-ionic active agent.

Another embodiment of the present invention provides a method of forming a vanishing cream suitable for administering a ionic active agent to animal or plant comprising, mixing:
a. from about 20 to about 50 wt. % water, based on the total weight of cream base composition;
b. at least about 30 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. an ionic active agent.

Another embodiment of the present invention provides for a vanishing cream composition suitable for topical application to an animal comprising:
a. from about 25 to 50 wt. % water, based on the total weight of cream base composition;
b. from about 25 to about 45 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. from about 0.5 wt. % to about 5 wt. % testosterone;
wherein said cream base is stable upon storage at ambient conditions, and clarifies at a temperature greater than 30° C.

Another embodiment of the present invention provides a vanishing cream composition suitable for topical application to an animal comprising:
a. from about 40 to about 50 wt. % water, based on the total weight of cream base composition;
b. from about 35 to about 50 wt. %, based on the total weight of cream base, of at least one alcohol;
c. at least one polymeric thickening agent comprising a nitrogen containing acrylic unit;
d. a penetration enhancing effective amount of a skin penetration enhancing_ compound;
e. an emulsifying agent; and
f. from about 0.5 wt. % to about 5 wt. % prostaglandin E1;
wherein said cream base is stable upon storage at ambient conditions, and clarifies at a temperature greater than 30° C.

Another embodiment of the present invention provides a vanishing cream composition suitable for topical application to an animal comprising:
a. from 0 to about 20 wt. % water, based on the total weight of cream base composition;
b. at least 60 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. from about 1 wt. % to about 5 wt. % hydroquinone;
wherein said cream base is stable upon storage at ambient conditions, and clarifies at a temperature greater than 30° C.

### BRIEF DESCRIPTION OF THE DRAWINGS

FIG. 1 shows a schematic representation of the diffusion cells used in Example 1.

FIGS. 2A and 2B show graphs illustrating average flux (FIG. 2A) and cumulative delivery (FIG. 2B) of PGE₁ across human skin vs. time for formulation number 11 in Table 1, against a clear gel control of 1% PGE₁, 5% SEPA-0009, 1% Klucel HF, 65.1% ethanol, and 27.9% water.

FIGS. 3A and 3B show graphs illustrating average flux (FIG. 3A) and cumulative delivery (FIG. 3B) of PGE₁ across human skin vs. time for formulations 11 and 13 in Table 1, against a clear gel control of 1% PGE₁, 5% SEPA-0009, 1% Klucel HF, 65.1% ethanol, and 27.9% water.

FIGS. 4A and 4B show graphs illustrating average flux (FIG. 4A) and cumulative delivery (FIG. 4B) of PGE, across human skin vs. time for formulations 11, 13 and 16 in Table 1, against a clear gel control of 1% PGE1, 5% SEPA-0009, 1% Klucel HF, 65.1% ethanol, and 27.9% water.

1. Active Agents

As used herein, the term “active agent” means any chemical or biological material suitable for administration, that produces a desired biological, pharmacological, or physiological effect in an animal or plant to which the agent is administered. Such effects may include, but are not limited to (1) having a prophylactic effect on the animal or plant, such as preventing an undesired biological effect, for example, as in preventing an infection; (2) alleviating a condition caused by a disease of the animal or plant, for example, alleviating pain or inflammation caused as a result of disease; and/or (3) either alleviating, reducing, or completely eliminating a disease from the animal or plant. The effect may be local, such as providing for a local anesthetic effect, or it may be systemic. Active agents are present in a pharmaceutically effective amount. The term “animal” as use herein is understood to also include human beings as well as other mammals.

Active agents that may be used in the compositions of the present invention include any locally or systemically active agents which are compatible with the compositions of the present invention and which can be delivered through the skin or other membrane to achieve a desired effect. In addition to pharmaceuticals, the present invention may also include other active agents, such as cosmetic agents. Representative active agents (grouped by therapeutic class) include but are not limited to:

Alimentary System

Antidiarrhoeals such as diphenoxylate, loperamide and hyoscyamine.

Cardiovascular System

Antihypertensives such as hydralazine, minoxidil, captopril, enalapril, clonidine, prazosin, debrisoquine, diazoxide, guanethidne, methyldopa, reserpine, trimetaphan.

Calcium channel blockers such as diltiazem, felodopine, amlodipine, nitrendipine, nifedipine and verapamil.

Antiarrhyrthmics such as amiodarone, flecainide, disopyramide, procainamide, mexiletene and quinidine.

Antiangina agents such as glyceryl trinitrate, erythritol tetranitrate, pentaerythritol tetranitrate, mannitol hexanitrate, perhexilene, isosorbide dinitrate and nicorandil.

Beta-adrenergic blocking agents such as alprenolol, atenolol, bupranolol, carteolol, labetalol, metoprolol, nadolol, nadoxolol, oxprenolol, pindolol, propranolol, sotalol, timolol and timolol maleate.

Cardiotonic glycosides such as digoxin and other cardiac glycosides and theophylline derivatives.

Adrenergic stimulants such as adrenaline, ephedrine, fenoterol, isoprenaline, orciprenaline, rimeterol, salbutamol, salmeterol, terbutaline, dobutamine, phenylephrine, phenylpropanolamine, pseudoephedrine and dopamine.

Vasodilators such as cyclandelate, isoxsuprine, papaverine, dipyrimadole, isosorbide dinitrate, phentolamine, nicotinyl alcohol, co-dergocrine, nicotinic acid, glyceryl trinitrate, pentaerythritol tetranitrate and xanthinol.

Antimigraine preparations such as ergotamine, dihydroergotamine, methysergide, pizotifen and sumatriptan.

Drugs Affecting Blood and Haemopoietic Tissues

Anticoagulants and thrombolytic agents such as warfarin, dicoumarol, low molecular weight heparins such as enoxaparin; streptokinase and its active derivatives. Haemostatic agents such as aprotinin, tranexamic acid and protamine.

Central Nervous System

Analgesics, antipyretics including the opiod analgesics such as buprenorphine, dextromoramide, dextropropoxyphene, fentanyl, alfentanil, sufentanil, hydromorphone, methadone, morphine, oxycodone, papaveretum, pentazocine, pethidine, phenoperidine, codeine and dihydrocodeine. Others include acetylsalicylic acid (aspirin), paracetamol, and phenazone.

Hypnotics and sedatives such as the barbiturates, amylobarbitone, butobarbitone and pentobarbitone and other hypnotics and sedatives such as choral hydrate, chlormethiazole, hydroxyzine and meprobamate.

Antianxiety agents such as the benzodiazepines, alprazolam, bromazepam, chlordiazepoxide, clobazam, chlorazepate, diazepam, flunitrazepam, flurazepam, lorazepam, nitrazepam, oxazepam, temazepam and triazolam.

Neuroleptic and antipsychotic drugs such as the phenothiazines, chlorpromazine, fluphenazine, pericyazine, perphenazine, promazine, thiopropazate, thioridazine and trifluoperazine and the butyrophenone, droperidol and haloperidol and the other antipsychotic drugs such as pimozide, thiothixene and lithium.

Antidepressants such as the tricyclic antidepressants amitryptyline, clomipramine, desipramine, dothiepin, doxepin, imipramine, nortriptyline, opipramol, protriptyline and trimipramine and the tetracyclic antidepressants such as mianserin and the monoamine oxidase inhibitors such as isocarboxazid, phenelizine, tranylcypromine and moclobemide and selective serotonin re-uptake inhibitors such as fluoxetine, paroxetine, citalopram, fluvoxamine and sertraline.

CNS stimulants such as caffeine.

Anti-alzheimer's agents such as tacrine.

Antiparkinson agents such as amantadine, benserazide, carbidopa, levodopa, benztropine, biperiden, benzhexol, procyclidine and dopamine-2 agonists such as S(−)-2-(N-propyl-N-2-thienylethylamino)-5-hydroxytetralin (N-0923).

Anticonvulsants such as phenytoin, valproic acid, primidone, phenobarbitone, methylphenobarbitone and carbamazepine, ethosuximide, methsuximide, phensuximide, sulthiame and clonazepam.

Antiemetics, antinauseants such as the phenothiazines, prochloperazine, thiethylperazine and 5HT-3 receptor antagonists such as ondansetron and granisetron and others such as dimenhydrinate, diphenhydramine, metoclopramide, domperidone, hyoscine, hyoscine hydrobromide, hyoscine hydrochloride, clebopride and brompride.

Musculoskeletal System

Non-steroidal anti-inflammatory agents including their racemic mixtures or individual enantiomers where applicable, such as ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol and ketoralac. Non-steroidal antiinflammatory agents may also include salicylamide, salicylic acid, flufenisal, salsalate, triethanolamine salicylate, aminopyrine, antipyrine, oxyphenbutazone, apazone, cintazone, flufenamic acid, clonixeril, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloide, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

Antirheumatoid agents such as penicillamine, aurothioglucose, sodium aurothiomalate, methotrexate and auranofin.

Muscle relaxants such as baclofen, diazepam, cyclobenzaprine hydrochloride, dantrolene, methocarbamol, orphenadrine and quinine.

Agents used in gout and hyperuricaemia such as allopurinol, colchicine, probenecid and sulphinpyrazone.

Hormones and Steroids

Oestrogens such as oestradiol, oestriol, oestrone, ethinyloestradiol, mestranol, stilboestrol, dienoestrol, epioestriol, estropipate and zeranol.

Progesterone and other progestagens such as allyloestrenol, dydrgesterone, lynoestrenol, norgestrel, norethyndrel, norethisterone, norethisterone acetate, gestodene, levonorgestrel, medroxyprogesterone and megestrol.

Antiandrogens such as cyproterone acetate and danazol.

Antioestrogens such as tamoxifen and epitiostanol and the aromatase inhibitors, exemestane and 4-hydroxy-androstenedione and its derivatives. Androgens and anabolic agents such as testosterone, methyltestosterone, clostebol acetate, drostanolone, furazabol, nandrolone oxandrolone, stanozolol, trenbolone acetate, dihydro-testosterone, 17-.alpha.-methyl-19-nortestosterone and fluoxymesterone.

5-alpha reductase inhibitors such as finasteride, turosteride, LY-191704 and MK-306.

Corticosteroids such as betamethasone, betamethasone valerate, cortisone, dexamethasone, dexamethasone 21-phosphate, fludrocortisone, flumethasone, fluocinonide, fluocinonide desonide, fluocinolone, fluocinolone acetonide, fluocortolone, halcinonide, halopredone, hydrocortisone, hydrocortisone 17-valerate, hydrocortisone 17-butyrate, hydrocortisone 21-acetate methylprednisolone, prednisolone, prednisolone 21-phosphate, prednisone, triamcinolone, triamcinolone acetonide.

Further examples of steroidal antiinflammatory agents include cortodoxone, fluoracetonide, fludrocortisone, difluorsone diacetate, flurandrenolone acetonide, medrysone, amcinafel, amcinafide, betamethasone and its other esters, chloroprednisone, clorcortelone, descinolone, desonide, dichlorisone, difluprednate, flucloronide, flumethasone, flunisolide, flucortolone, fluoromethalone, fluperolone, fluprednisolone, meprednisone, methylmeprednisolone, paramethasone, cortisone acetate, hydrocortisone, cyclopentylpropionate, cortodoxone, flucetonide, fludrocortisone acetate, flurandrenolone acetonide, medrysone, amcinafal, amcinafide, betamethasone, betamethasone benzoate, chloroprednisone acetate, clocortolone acetate, descinolone acetonide, desoximetasone, dichlorisone acetate, difluprednate, flucloronide, flumethasone pivalate, flunisolide acetate, fluperolone acetate, fluprednisolone valerate, paramethasone acetate, prednisolamate, prednival, triamcinolone hexacetonide, cortivazol, formocortal and nivazol.

Pituitary hormones and their active derivatives or analogs such as corticotrophin, thyrotropin, follicle stimulating hormone (FSH), luteinising hormone (LH) and gonadotrophin releasing hormone (GnRH).

Hypoglycemic agents such as insulin, chlorpropamide, glibenclamide, gliclazide, glipizide, tolazamide, tolbutamide and metformin.

Thyroid hormones such as calcitonin, thyroxine and liothyronine and antithyroid agents such as carbimazole and propylthiouracil.

Other miscellaneous hormone agents such as octreotide.

Pituitary inhibitors such as bromocriptine.

Ovulation inducers such as clomiphene.

Genitourinary System

Diuretics such as the thiazides, related diuretics and loop diuretics, bendrofluazide, chlorothiazide, chlorthalidone, dopamine, cyclopenthiazide, hydrochlorothiazide, indapamide, mefruside, methycholthiazide, metolazone, quinethazone, bumetanide, ethacrynic acid and frusemide and potassium sparing diuretics, spironolactone, amiloride and triamterene.

Antidiuretics such as desmopressin, lypressin and vasopressin including their active derivatives or analogs.

Obstetric drugs including agents acting on the uterus such as ergometrine, oxytocin and gemeprost.

Prostaglandins such as alprostadil (PGE1), prostacyclin (PGI2), dinoprost (prostaglandin F2-alpha) and misoprostol.

Antimicrobials

Antimicrobials including the cephalosporins such as cephalexin, cefoxytin and cephalothin.

Penicillins such as amoxycillin, amoxycillin with clavulanic acid, ampicillin, bacampicillin, benzathine penicillin, benzylpenicillin, carbenicillin, cloxacillin, methicillin, phenethicillin, phenoxymethylpenicillin, flucloxacillin, mezlocillin, piperacillin, ticarcillin and azlocillin.

Tetracyclines such as minocycline, chlortetracycline, tetracycline, demeclocycline, doxycycline, methacycline and oxytetracycline and other tetracycline-type antibiotics.

Aminoglycosides such as amikacin, gentamicin, kanamycin, neomycin, netilmicin and tobramycin. Antifungals such as amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione.

Quinolones such as nalidixic acid, cinoxacin, ciprofloxacin, enoxacin and norfloxacin. Sulphonamides such as phthalylsulphthiazole, sulfadoxine, sulphadiazine, sulphamethizole and sulphamethoxazole.

Sulphones such as dapsone.

Other miscellaneous antibiotics such as chloramphenicol, clindamycin, erythromycin, erythromycin ethyl carbonate, erythromycin estolate, erythromycin glucepate, erythromycin ethylsuccinate, erythromycin lactobionate, roxithromycin, lincomycin, natamycin, nitrofurantoin, spectinomycin, vancomycin, aztreonam, colistin IV, metronidazole, tinidazole, fusidic acid and trimethoprim; 2-thiopyridine N-oxide; halogen compounds, particularly iodine and iodine compounds such as iodine-PVP complex and diiodohydroxyquin; hexachlorophene; chlorhexidine; chloroamine compounds; benzoylperoxide.

Antituberculosis drugs such as ethambutol, isoniazid, pyrazinamide, rifampicin and clofazimine. Antimalarials such as primaquine, pyrimethamine, chloroquine, hydroxychloroquine, quinine, mefloquine and halofantrine.

Antiviral agents such as acyclovir and acyclovir prodrugs, famciclovir, zidovudine, didanosine, stavudine, lamivudine, zalcitabine, saquinavir, indinavir, ritonavir, n-docosanol, tromantadine and idoxuridine.

Anthelmintics such as mebendazole, thiabendazole, niclosamide, praziquantel, pyrantel embonate and diethylcarbamazine.

Cytotoxic agents such as plicamycin, cyclophosphamide, dacarbazine, fluorouracil and its prodrugs [described,for example, in International Journal of Pharmaceutics 111, 223-233 (1994)], methotrexate, procarbazine, 6-mercaptopurine and mucophenolic acid.

Metabolism

Anorectic and weight reducing agents including dexfenfluramine, fenfluramine, diethylpropion, mazindol and phentermine.

Agents used in hypercalcaemia such as calcitriol, dihydrotachysterol and their active derivatives or analogs.

Respiratory System

Antitussives such as ethylmorphine, dextromethorphan and pholcodine.

Expectorants such as acetylcysteine, bromhexine, emetine, guaiphenesin, ipecacuanha and saponins.

Decongestants such as phenylephrine, phenylpropanolamine and pseudoephedrine.

Antiasthmatic agents such as terbutaline.

Bronchospasm relaxants such as ephedrine, fenoterol, orciprenaline, rimiterol, salbutamol, sodium cromoglycate, cromoglycic acid and its prodrugs [described, for example, in International Journal of Pharmaceutics 7, 63-75 (1980)], terbutaline, ipratropium bromide, salmeterol and theophylline and theophylline derivatives.

Allergy and Immune System

Antihistamines such as meclozine, cyclizine, chlorcyclizine, hydroxyzine, brompheniramine, chlorpheniramine, clemastine, cyproheptadine, dexchlorpheniramine, diphenhydramine, diphenylamine, doxylamine, mebhydrolin, pheniramine, tripolidine, azatadine, diphenylpyraline, methdilazine, terfenadine, astemizole, loratidine and cetirizine.

Local anaesthetics such as bupivacaine, amethocaine, lignocaine, cinchocaine, dibucaine, mepivacaine, prilocaine and etidocaine.

Stratum corneum lipids, such as ceramides, cholesterol and free fatty acids, for improved skin barrier repair. See: Man, et al., J. Invest. Dermatol., 106(5), 1096 (1996).

Neuromuscular blocking agents such suxamethonium, alcuronium, pancuronium, atracurium, gallamine, tubocurarine and vecuronium.

Smoking cessation agents such as nicotine, bupropion and ibogaine.

Insecticides and other pesticides which are suitable for local or systemic application to plants.

Dermatological agents, such as vitamins A and E, vitamin E acetate and vitamin E sorbate.

Allergens for desensitisation such as house dust mite allergen.

Nutritional agents, such as vitamins, essential amino acids and essential fats.

Keratolytics such as the alpha-hydroxy acids, glycollic acid and salicylic acid.

Psychicenergisers, such as 3-(2-aminopropyl)indole, 3-(2-aminobutyl)indole, and the like.

Anti-acne agents such as containing isotretinoin, tretinoin and benzoyl peroxide.

Anti-psoriasis agents such as containing etretinate, cyclosporin and calcipotriol.

Anti-itch agents such as capsaicin and its derivatives such as nonivamide

[Tsai, et al., Drug. Dev. Ind Pharm., 20(4), 719 (1994)].

Anticholinergic agents, which are effective for the inhibition of axillary sweating and for the control of prickly heat. The antiperspirrant activity of agents such as methatropine nitrate, propantheline bromide, scopolamine, methscopolamine bromide, and the new class of soft antiperspirants, quaternary acyloxymethyl ammonium salts [described, for example, by Bodor, et al., J. Med. Chem. 23, 474 (1980) and also in United Kingdom Specification No. 2010270, published Jun. 27, 1979].

Physiologically active peptides and proteins. Specific-examples of peptides and proteins include, human growth hormone, LHRH, LHRH analogs such as goserelin, buserelin, gonadorelin, napharelin and leuprolide, GHRH, GHRF, insulin, insultropin, calcitonin, octreotide, endorphin, TRH, NT-36 (chemical name: [[(s)4-oxo-2-azetidinyl] carbonyl]-L-histidyl-L-prolinamide), liprecin, pituitary hormones (e.g., HGH, HMG, desmopressin acetate, etc), follicle luteoids, alpha-ANF, growth factors such as growth factor releasing factor (GFRF), beta-MSH, somatostatin, bradykinin, somatotropin, platelet-derived growth factor, asparaginase, bleomycin sulfate, chymopapain cholecystokinin, chorionic gonadotropin, corticotropin (ACTH), erythropoietin, epoprostenol (platelet aggregation inhibitor), glucagon, HCG, hirulog, hyaluronidase, interferon, interleukins, menotropins (urofollitropin (FSH) and LH), oxytocin, streptokinase, tissue plasminogen activator, urokinase, vasopressin, desmopressin, ACTH analogs, ANP, ANP clearance inhibitors, angiotensin II antagonists, antidiuretic hormone agonists, bradykinin antagonists, CD4, ceredase, CSI's , enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vaccines, vasopressin antagonists analogs, alpha-1 antitrypsin (recombinant), and TGF-beta.

The active agents of the present invention may be either ionic or non-ionic.

These active agents may be used as such or in the form of salts, esters or prodrugs. Prodrugs include esters, amides, or other derivatives of active agents which may generate the active agent in vivo upon administration. Different drugs may have multiple physiological, pharmacological or cosmetic effects, which, as known to those skilled in the art, may vary as a function of concentration.

The active agents may be present in the compositions in pharmacologically, pharmaceutically or cosmetically effective amounts and will depend on such factors as the disease or condition being treated, the age of the patient and other factors well understood by those skilled in the art. Generally, amounts of active agent may range from about 0.01 wt. % to about 15 wt. % relative to the weight of the total composition, such as from about 0.1 wt. % to about 12% wt, such as from about 0.5 wt. % to about 5 wt. %, or from about 1 wt. % to about 10 wt. %, or from about 1 wt. % to about 5 wt. %, for example, from about 1.5 wt. % to about 3 wt. % by weight of the composition.

2. Cream Base Formulations

The present invention also provides cream base formulations for delivering one or more active agents to a plant or animal. Such cream compositions resist separation under ambient conditions, and maintain a pleasant consistency for application. Such cream base formulations may deliver a non-ionic active agent, an ionic active agent, or a mixture of ionic and non-ionic active agents.

Traditionally, cosmetic or pharmaceutical creams were limited to oil-in-water emulsions which contain more than 50 wt. % of water. According to embodiments of the present invention, stable creams may include those with less than 50 wt. % water, by the use of certain polymeric thickening agents. Such cream formulations have the advantage of being able to incorporate larger quantities of organic solvents, such as alcohols (e.g., ethanol) while still maintaining acceptable cream properties. Creams which have, in the past, incorporated skin penetration enhancing agents have been typically limited to low concentrations of these compounds (e.g., <5 wt. %) because higher concentrations of such skin penetration enhancers resulted in either emulsion destabilization, or required more organic solvents, such as alcohols, which will breakdown the cream. Therefore, the creams of the present invention provide, inter alia, for creams with higher concentrations of skin penetration enhancers, while still maintaining an acceptable cream feel.

The term “cream” as used herein refers to a composition which is opaque or milky at room temperature and is an oil in water emulsion with a hydrophilic phase in which a hydrophobic phase is emulsified and dispersed, in a micellar structure. Cream compositions of the present invention may include those cream compositions which clarify or liquefy when contacted with animal, e.g., human, skin so as to not discolor or otherwise present an unpleasant aesthetic quality to the user. Such “vanishing creams” typically will clarify at temperatures above ambient temperature, for example, above about 30° C., such as above about 33° C., or above about 35° C. These qualities of a vanishing cream are thought to occur by the thermal breakdown of the micellar structure of the cream at elevated temperature, resulting in the separation of the emulsion, and loss of opacity.

The components of the cream base formulations of the present invention may include a hydrophobic component, hydrophilic component, and a stabilizing effective amount of a polymeric stabilizing agent. Optionally, the cream base formulations may further comprise an emulsifying agent and a skin penetration enhancing agent. The active agent of the present invention may be dispersed, dissolved or suspended within the cream base formulation of the present invention, for example, in one or both the hydrophobic or hydrophilic components.

a. Hydrophilic Component

Compositions of the present invention include a hydrophilic component, e.g., water and/or other water soluble or water miscible compounds. Suitable water soluble or dispersible ingredients may comprise alcohols, carboxylic acids, diols, triols, polyols and the like.

According to one embodiment of the present invention, the hydrophilic component includes water and one or more alcohols. Suitable alcohols in the present invention include: straight or branched chain alkyl, aromatic, or alkylene alcohols. In one embodiment, the alcohol used in the hydrophilic component is a linear alkyl alcohol containing between 1 and 6 carbon atoms, for example, methanol, ethanol, n-propanol, iso-propanol, n-butanol, iso-butanol, tert-butanol, 1-pentanol, or 1-hexananol. In another embodiment, the alcohol is methanol or ethanol. Polyols, including diols and triols may be present in place of or together with the monoalcohol. Representative polyols include, for example, ethylene glycol, propylene glycol, diethylene glycol, butylene glycol, and the like. The amount of alcohol used in the hydrophilic component may be selected to accomplish the emulsions described herein, and may generally comprise between 1 and 99 wt. % relative to the total weight of the hydrophilic component. The alcohol may constitute between about 15 and about 85 wt. % of the hydrophilic component, such as between about 30 and about 70 wt. %, for example between about 35 and about 55 wt. % of the hydrophilic component.

The amount of the hydrophilic component present in the present invention will depend on the additional components present in the composition as described herein. The compositions of the present invention will include between about 40 and about 90 wt. % relative to the total weight of the hydrophilic component. In one embodiment, the compositions of the present invention will include between about 45 and about 85 wt. % of the hydrophilic component, such as between about 50 and about 80 wt. %, or between about 60 to about 70 wt. %.

For example, in one embodiment, the hydrophilic phase comprises water in at least about 5 wt. %, for example, at least about 10 wt. %, such at least about 15 wt. %, at least about 20 wt. %, at least about 25 wt. %, at least about 30 wt. %, at least about 40 wt. %, at least about 45 wt. %, at least about 50 wt. %, at least about 55 wt. %, at least about 60 wt. % or least about 70 wt. %, based on the total weight of the composition. The amount of water may be less than about 85 wt. % based on the total weight of the composition, for example, less than about 80 wt. %, such as less than about 75 wt. %, less than about 70 wt. %, less than about 65 wt. %, less than about 60 wt. %, less than about 50 wt. %, less than about 55 wt. %, less than about 50 wt. %, less than about 45 wt. %, less than about 40 wt. % or less than about 35 wt. % water.

The hydrophilic phase may comprise water between about 15 to about 89 wt. %, for example, between about 20 and about 80 wt, or from about 25 to about 70 wt. %, or from about 30 to about 60 wt. % by weight of the composition.

In one embodiment, ethanol may be present in at least 15 wt. % relative to the total weight of the composition, for example, in at least about 20 wt. %, such as at least about 25 wt. %, at least about 30 wt. %, at least about 40 wt. %, at least about 45 wt. %, at least about 50 wt. %, at least about 55 wt. %, or at least about 60 wt. %. The amount of ethanol may be less than, for example less than about 65 wt. %, such as less than about 60 wt. %, or less than about 55 wt. %, or less than about 50 wt. %, or less than about 45 wt. %, or less than about 40 wt. %, or less than about 35 wt. %.

b. Hydrophobic component

The hydrophobic component of the present invention may include an oily or fatty component, comprising from about 5 to about 60 wt. % of the total weight of the composition, for example, from about 9 to about 50 wt. % of the total weight of the composition, such as about 12 to about 40 wt. % of the total weight of the composition, such as about 15 to about 30 wt. % of the total weight of the composition.

This fatty component may include one or more oils which may be selected from: volatile or nonvolatile silicones which are linear, branched or cyclic, organomodified or otherwise water-insoluble or fat-soluble, mineral oils such as paraffin oil and liquid petroleum jelly; oils of animal origin such as perhydrosqualene; oils of plant origin such as sweet almond oil, avocado oil, castor oil, olive oil, jojoba oil, sesame oil, groundnut oil, macadamia oil, grape seed oil, rapeseed oil, copra oil; synthetic oils such as isoparaffins, fluorinated and perfluorinated oils; and fatty acid esters.

The hydrophobic component may be substantially or essentially water insoluble and may be derived from animals, plants, or petroleum and may be natural or synthetic (i.e., man-made) or semi synthetic.

Non-limiting examples of suitable hydrophobic components include the following representative materials:
(1) Mineral oil, also known as petrolatum liquid, a mixture of liquid hydrocarbons obtained from petroleum. See: The Merck Index, Tenth Edition, Entry 7048, p. 1033 (1983), and International Cosmetic Ingredient Dictionary, Fifth Edition, vol. 1, p.415-417 (1993), which are incorporated by reference herein in their entirety.
(2) Petrolatum, also known as petroleum jelly, a colloidal system of branched solid hydrocarbons and high-boiling liquid hydrocarbons, in which most of the liquid hydrocarbons are believed to be held inside micelles. See: The Merck Index, Tenth Edition, Entry 7047, p. 1033 (1983); Schindler, Drug. Cosmet. Ind., 89, 36-37, 76, 78-80, 82 (1961); and International Cosmetic Ingredient Dictionary, Fifth Edition, vol. 1, p. 537 (1993), which are incorporated by reference herein in their entirety.
(3) Straight and branched chain hydrocarbons having from about 7 to about 40 carbon atoms. For example, dodecane, isododecane, squalane, cholesterol, hydrogenated polyisobutylene, docosane (i.e., a C₂₂ hydrocarbon), hexadecane, isohexadecane (a commercially available hydrocarbon sold as Permethyl® 101A by Presperse, South Plainfield, N.J.). Also useful are the C₇-C₄₀ isoparaffins, which are C₇₋₄₀ branched hydrocarbons.
(4) C₁-C₃₀ alcohol esters of C₁-C₃₀ carboxylic acids and of C₂-C₃₀ dicarboxylic acids, including straight and branched chain materials as well as aromatic derivatives (as used herein in reference to the hydrophobic component, mono- and poly-carboxylic acids include straight chain, branched chain and aryl carboxylic acids). Non-limiting examples include diisopropyl sebacate, diisopropyl adipate, isopropyl myristate, isopropyl palmitate, methyl palmitate, myristyl propionate, 2-ethylhexyl palmitate, isodecyl neopentanoate, di-2-ethylhexyl maleate, cetyl palmitate, myristyl myristate, stearyl stearate, isopropyl stearate, methyl stearate, cetyl stearate, behenyl behenate, dioctyl maleate, dioctyl sebacate, diisopropyl adipate, cetyl octanoate, diisopropyl dilinoleate.
(5) mono-, di- and tri-glycerides of C₁-C₃₀ carboxylic acids, e.g., caprylic/capric triglyceride, PEG-6 caprylic/capric triglyceride, PEG-8 caprylic/capric triglyceride.
(6) alkylene glycol esters of C₁-C₃₀ carboxylic acids, e.g., ethylene glycol mono- and di-esters, and propylene glycol mono- and di-esters of C₁-C₃₀ carboxylic acids e.g., ethylene glycol distearates.
(7) propoxylated and ethoxylated derivatives of the foregoing materials that are substantially hydrophobic.
(8) C₁-C₃₀ mono- and poly-esters of sugars and related materials. These esters may be derived from a sugar or polyol moiety and one or more carboxylic be in either liquid or solid form at room temperature. Examples of liquid esters include: glucose tetraoleate, the glucose tetraesters of soybean oil fatty acids, the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoleate, sucrose hexaoleate, sucrose hepatoleate, sucrose octaoleate, and mixtures thereof.
(9) Organopolysiloxane oils. The organopolysiloxane oil may be volatile, non-volatile, or a mixture of volatile and non-volatile silicones. Suitable organopolysiloxanes can be selected from a wide variety of silicones spanning a broad range of volatilities and viscosities. Nonlimiting examples of suitable silicones are disclosed in U.S. Pat. No. 5,069,897, to Orr, issued Dec. 3, 1991, which is incorporated by reference herein in its entirety. Examples of suitable organopolysiloxane oils include polyalkylsiloxanes, cyclic polyalkylsiloxanes, and polyalkylarylsiloxanes. Organopolysiloxanes may also be selected from polyalkylsiloxanes, alkyl substituted dimethicones, cyclomethicones, trimethylsiloxysilicates, dimethiconols, polyalkylaryl siloxanes, and mixtures thereof. In one embodiment, the polyalkylsiloxanes are dimethicones.
(10) Vegetable oils and hydrogenated vegetable oils. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.
(11) animal fats and oils, e.g., lanolin and derivatives thereof, cod liver oil.
(12) Also useful are C₄-C₂₀ alkyl ethers of polypropylene glycols, C₁-C₂₀ carboxylic acid esters of polypropylene glycols, and di-C₈-C₃₀ alkyl ethers. Nonlimiting examples of these materials include PPG-14 butyl ether, PPG-15 stearyl ether, dioctyl ether, dodecyl octyl ether, and mixtures thereof.
(13) The hydrophobic component may also comprise one or more fatty alcohols, fatty acids or waxes (such as, for example, paraffin, polyethylene wax, Carnauba wax, beeswax).
   c. Polymeric Thickening Agents

Suitable polymeric thickening agents include those polymers that comprise a nitrogen containing acrylic unit. Examples of the nitrogen containing acrylic units include those derived from monomers of acrylamide, acryloyltaurate, or other arylamides. Examples of polymers comprising a nitrogen containing acrylic unit include an ionic polyamide polymer containing acrylamidopropanesulfonic acid (AMPS) and/or its salts as a comonomer. These polymers can be formed from a variety of monomers including acrylamide and methacrylamide, which may be unsubstituted or substituted with one or two alkyl groups (such as C₁ to C₅), or N-vinyl pyrrolidone. In one embodiment the acrylate amide and methacrylate amide are monomers in which the amide nitrogen is unsubstituted, or substituted with one or two C₁ to C₅ alkyl groups (preferably methyl, ethyl, or propyl), for example, acrylamide, methacrylamide, N-methacrylamide, N-methylmethacrylamide, N,N-dimethylmethacrylamide, N-isopropylacrylamide, N-isopropylmethacrylamide, and N,N-dimethylacrylamide. Specific examples of the polymeric thickening agents include acrylamide-acryloyldimethyltaurate or acrylamide-acryloyldimethlaurate or copolymers (e.g., Simulgel 600 available from Seppic, Paris, France), polyacrylamides (e.g., Sepigel 305 available from Seppic, Paris, France), sodium acrylate-sodium acryloyidimethyl laurate or sodium acrylate-sodium acryloyidimethyl taurate copolymers (e.g.; Simugel EG available from Seppic, Paris, France), and ammonium acryloyidimethyllaurate-Beheneth-25 methacrylate cross polymers (e.g., Aristoflex available from Clariant Corporation, Charlotte, N.C.). Mixtures of two or more polymeric thickening agents may also be used.

The polymeric thickening agents of the present invention are included in an amount sufficient to prevent visible separation of the composition for at least seven days when stored at ambient conditions (i.e., room temperature and pressure), after which time, no more than 10% of the active agent or the optional skin penetration enhancer have degraded, or otherwise reacted. The polymeric thickening agents are generally present in a concentration of about 0.1 to about 10 wt. %. One of skill in the art will also recognize that the amount of polymeric thickening agent necessary will depend upon the hydrophobic and hydrophilic phases, intended use, intended storage and use conditions, and other optional ingredients which may be used within the composition as well as the mixing conditions and mixing apparatus used to prepare the emulsion or dispersion.

In one embodiment, the polymeric thickening agent is present from between about 1 and about 10 wt. % , for example, from about 0.2 to about 10 wt. %, such as form about 2 to about 10 wt. %, such as from about 2 to about 8 wt. %, or from about 3 to about 7 wt. %, such as about 4 to about 6 wt. %. In another embodiment, the polymeric thickening agent is present in an amount of at least about 0.2 wt. %, based on the total weight of the composition, for example, at least about 0.5 wt. %, for example, at least about 1 wt. %, at least about 2 wt. %, at least about 3 wt. %, at least about 4 wt. %, or at least 5 wt. %.

d. Skin Penetration Enhancing Compounds

In one embodiment a skin penetration enhancing compound is optionally used to promote permeation of an active agent through the skin. A skin penetration. enhancing effective amount of the skin penetration enhancing (SPE) compound may be determined by review of the literature for the particular enhancer or otherwise determined by routine in vitro and/or in vivo studies. Skin penetration enhancers containing a C₆ to C₁₂ alkyl group are may be useful in the cream compositions of the present invention.

In one embodiment the skin penetration enhancing compound is a 2-hydrocarbyl group substituted 1,3-dioxolane of the formula (I): 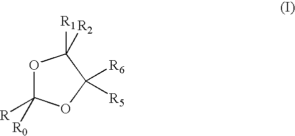
or a 1,3-dioxane of the formula (II): 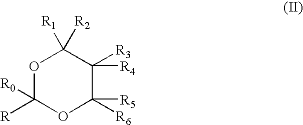
or an acetal (including herniacetal) of the formula (III): 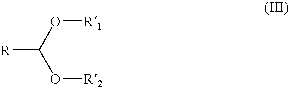
where R represents a C₆ to C₂₀ aliphatic group, R₀, R₁, R₂, R₃, R₄, R₅, and R₆, each, independently, represent hydrogen or a C₁ to C₄ aliphatic group; R′₁, and R′₂, each, independently, represent C₁ to C₄ aliphatic group. Several compounds of these formulas are available commercially from MacroChem Corporation under the trademark SEPA®.

In one particular embodiment, R represents a C₆ to C₁₂ aliphatic group; especially C₇ to C₁₀ aliphatic group. The aliphatic group may be a straight or branched chain alkyl or alkenyl group, such as, for example, n-hexyl, n-heptyl, n-octyl, n-nonyl, n-decyl, n-undecyl, n-dodecyl, 2-methyl-octyl, 4-ethyl-decyl, and the like.

The C₁ to C₄ aliphatic group may be, for example, methyl, ethyl, n-propyl, isopropyl, n-butyl, tert-butyl, ethenyl, and the like. In one embodiment aliphatic groups for R₁ to R₆ and for R′₁ and R′₂ are alkyl groups, especially alkyl having 1 or 2 carbon atoms, most especially ethyl. R₁ to R₆ may also all be hydrogen.

Specific enhancer compounds (i) include, for example, 2-n-heptyl-1,3-dioxolane, 2-n-nonyl-1,3-dioxolane, 2-n-undecyl-1,3-dioxolane, 2-n-nonyl-1,3-dioxane, 2-n-undecyl-1,3-dioxane, 2-n-heptylaldehyde-acetal, 2-n-octyl-aldehyde-acetal, 2-n-nonylaldehyde-acetal, 2-n-decylaldehyde-acetal, 3,7-dimethyl-2,6-octadienal (citral), citronal and the like. 2-n-nonyl-1,3-dioxolane (2-NND) is commercially available as SEPA-0009 from MacroChem Corporation.

Another class of SPE compounds (ii) are cyclic ketones and cyclic lactones and derivatives thereof, as disclosed in, for example, U.S. Pat. Nos. 5,023,252 and 5,731,303, the disclosures of which, are incorporated herein, in their entireties, by reference thereto.

The SPE compounds (ii) may be represented by the following formula (IV): 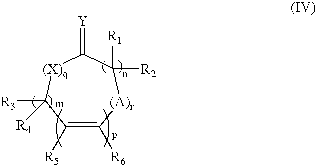
wherein X and Y are oxygen, sulfur or an imino group of the structure 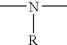
or ═N—R, with the proviso that when Y is the imino group, X is an imino group, and when Y is sulfur, X is sulfur or an imino group, A is group having the structure 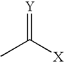
wherein X and Y are defined above,
m and n are integers having a value from 1 to 20 and the sum of m+n is not greater than 25,
p is an integer having a value of 0 or 1,
q is an integer having a value of 0 or 1,
r is an integer having a value of.0 or 1,
R represents hydrogen or a straight or branched chain alkyl group having from 1 to 6 carbon atoms, and,
R₁, R₂, R₃, R₄, R₅ and R₆, each, independently, represent hydrogen or a straight or branched chain alkyl group having from 1 to 6 carbon atoms, with the proviso that only one of R₁ to R₆ may be said alkyl group, and with the further provisos that,
when p, q and r have a value of 0 and Y is oxygen, m+n is at least 11,
when X is an imino group, q equals 1, Y is oxygen, and p and r are 0, then m+n is at least 11.

Examples of the alkyl group for R and R₁ to R₆ include methyl, ethyl, n-propyl, isopropyl, n-butyl, isobutyl, sec-butyl, amyl, hexyl, and the like.

Preferably, each of R and R₁ to R₆ are hydrogen atoms and X and Y each represent oxygen. These preferred compounds of formula (IV) are, therefore, cyclic ketones (when q and r are each 0) or cyclic lactones.

Another preferred class of compounds of formula (IV) may be represented by the following general formula (IV-A): 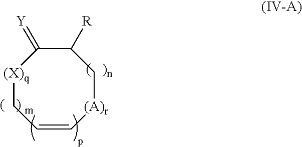
wherein X, Y, R, A, m, n, p, q and r, are as defined above.

Preferably, in formula (IV-A), X and Y are each oxygen and R is preferably hydrogen.

Pentadecalactone is especially preferred as the SPE of type (ii).

Another class of SPE compounds (iii) include an alkyl-2-(N,N-disubstituted amino)-alkanoate, an (N,N-disubstituted amino)-alkanol alkanoate, or a mixture of these, as more fully described in U.S. Pat. No. 6,046,244, the disclosure of which is incorporated herein by reference thereto. For convenient reference, alkyl-2-(N,N-disubstituted amino)-alkanoates and (N,N-disubstituted amino)-alkanol alkanoates can be grouped together under the label alkyl (N,N-disubstituted amino) esters.

Alkyl-2-(N,N-disubstituted amino)-alkanoates suitable for the present invention can be represented by the following formula (V) 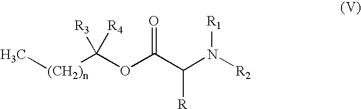
wherein n is an integer having a value in the range of about 4 to about 12;
R is a member of the group consisting of hydrogen, C₁ to C₇ alkyl, benzyl and phenyl;
R₁ and R₂ are members of the group consisting of hydrogen and C₁ to C₇ alkyl; and R₃ and R₄ are members of the group consisting of hydrogen, methyl and ethyl.

Preferred alkyl (N,N-disubstituted amino)-alkanoates are C₄ to C₁₂ alkyl (N,N-disubstituted amino)-acetates and C₄ to C₁₂ alkyl (N,N-disubstituted amino)-propionates. Exemplary specific alkyl-2-(N,N-disubstituted amino)-alkanoates include dodecyl 2-(N,N dimethylamino)-propionate (DDAIP); and dodecyl 2-(N,N-dimethylamino)-acetate (DDAA).

Alkyl-2-(N,N-disubstituted amino)-alkanoates are known. For example, dodecyl 2-(N,N-dimethylamino)-propionate (DDAIP) is available from Steroids, Ltd. (Chicago, Ill.). In addition, alkyl-2-(N,N-disubstituted amino)-alkanoates can be synthesized from more readily available compounds as described in U.S. Pat. No. 4,980,378 to Wong et al., which syntheses procedures are incorporated herein by reference.

Suitable (N,N-disubstituted amino)-alkanol alkanoates can be represented by the formula (VI): 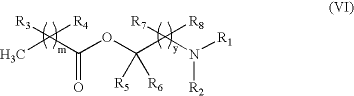
wherein m is an integer having a value in the range of about 5 to about 22, preferably, from about 5 to about 18; y is an integer having a value in the range of 0 to about 5; and R₁, R₂, R₃, R₄, R₅, R₆, and R₇ are members of the group consisting of hydrogen, C₁ to C₈ alkyl, and C₆ to C₈ aryl; and R₈ represents hydrogen, hydroxyl, C₁ to C₈ alkyl, or C₆ to C8 aryl.

Preferred (N,N-disubstituted amino)alkanol alkanoates include C₅ to C₁₈ carboxylic acid esters, such as the compounds of the following formula (VI-1): 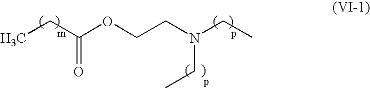
where m′ is an integer of from about 5 to about 21, preferably, from about 5 to about 16; and p is an integer of from 0 to about 3, preferably, 0 or 1, especially 0.

Exemplary specific alkyl alkanoate compounds of formula (V) include 1-(N,N-dimethylamino)-2-propanol dodecanoate (DAIPD), 1-(N,N-dimethylamino)-2-propanol myristate (DAIPM), and 1-(N,N-dimethylamino)-2-propanol oleate (DAIPO).

Among the suitable penetration enhancers for the present invention DDAIP and DAIPD may be specifically mentioned.

Another class of penetration enhancers of type (iv) include N-alkyl lactams, such as those disclosed in, for example, U.S. Pat. Nos. 4,316,893 and 4,424,210, the disclosures of which are incorporated herein, in their entirety, by reference thereto; and N-alkylazacycloheptanes, such as those disclosed in, for example, U.S. Pat. No. 5,204,339, the disclosure of which is incorporated herein, in its entirety, by reference thereto.

The N-alkyl lactams include, for example, compounds of the following formula (VII): 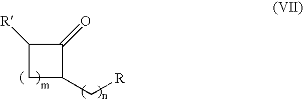
where R′ is H or a C₁ to C₄ alkyl group, R is C₁ to C₂ alkyl, phenyl or substituted phenyl, or the group 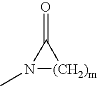
   m is an integer of 3 to 7, n is 0 or an integer of 1 to 17, except that when m is 3, n is from 7 to 17, and R is preferably methyl. In one embodiment, n is an integer such that the total number of carbon atoms represented by (CH₂)ₙ and R is from about 6 to 12.

A preferred class of lactams are represented by the following formula (VII-1) 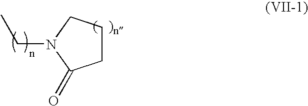
where n=0 or an integer from 1 to 10, and n″=0, 1 or 2.

Typical examples of compounds of formula (VII) include:
1-n-hexylazacyclopentan-2-one
1-n-heptaylazacyclopentan-2-one
1-n-octylazacyclopentan-2-one
1-n-nonylazacyclopentan-2-one
1-decylazacyclopentan-2-one
1-n-dodecylazacyclopentan-2-one
1-methylazacycloheptan-2-one
1-n-propylazacycloheptan-2-one
1-n-butylazacycloheptan-2-one
1-n-octylazacycloheptan-2-one
1-phenylazacyclopentan-2-one
1-(2-chlorophenyl)azacyclopentan-2-one
1,3-bis-(1-azacyclopentan-2-onyl)propane.

Of these, 1-n-dodecyl-azacycloheptan-2-one, is commercially available under the tradename, AZONE.

The N-alkylazacycloheptanes may be represented by the following formula (VIII): 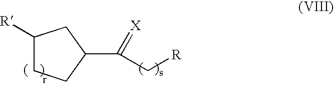
where X represents O or S, preferably O, R′ represents H or C₁ to C₄ alkyl; r is an integer of from 2 to 6, and s is 0 or an integer of 1 to 17.

Representative compounds of formula (VIII) include:
1-n-undecylformylazacycloheptane
1-n-decylformylazacycloheptane
1-n-octylformylazacycloheptane
1-n-nonylformylazacycloheptane
1-n-dodecylformylazacycloheptane
1-n-tetradecylformylazacycloheptane
1-n-hexadecylformylazacycloheptane
1-n-pentadecylformylazacycloheptane
1-n-heptadecylformylazacycloheptane
1-(16-methylhexadecyl)formylazacycloheptane.

Other skin penetration enhancing compounds useful in the present invention include alkyl esters.

In one embodiment, the skin penetration enhancing compounds may be present from 0 to 25 wt. %, for example, from about 5 to about 20 wt. %, such as from about 7 to about 15 wt. %, or from about 1 to about 25 wt. %, such as from about 2 to about 20 wt. %, or from about 3 to about 15 wt. % percent by weight of the composition.

e. Emulsifying Agents

An optional emulsifying agent, when present in an emulsifying effective amount, may be used to assist the hydrophobic and hydrophilic components in dispersing and forming an essentially uniform micellar composition with an appropriate cream consistency. A wide variety of emulsifying agents may be used. Any emulsifying agent will be suitable so long as it effectively forms the desired emulsion and does not react with, or prevent delivery of, the active agents. Emulsifying agents may be ionic or neutral.

Examples of suitable emulsifying agents include, disodium cocoamphodiacetate, oxyethylenated glyceryl cocoate (7 EO) such as the product sold by COGNIS under the name Cetiol HE, PEG-20 hexadecenyl succinate, PEG-15 stearyl ether; the ricinoleic monoethanolamide monosulfosuccinate salts such as the product sold by Goldschmidt under the name REWODERM S1333, oxyethylenated hydrogenated ricinoleic triglyceride containing 60 ethylene oxide units such as the product sold by Nikko under the name NIKKOL HCO-60 or such as the product sold by BASF under the name CREMOPHOR RH60 or CREMOPHOR RH 40 (polyoxyl 40 hydrogentate castor oil), polymers such as Poloxamers, which are block copolymers of ethylene oxide and propylene oxide, such as for example the product sold under the name Lutrol F68 by BASF and Poloxamer 407 sold under the name SYNPERONIC PE/F 127 by UNIQEMA The nonsolid fatty substances at room temperature (that is to say at a temperature ranging from about 20 to 35° C. such as sesame oil, sweet almond oil, apricot stone oil, sunflower oil, octoxyglyceryl palmitate (or 2-ethylhexyl glyceryl ether palmitate) such as the product marketed under the name Mexanyl GP by the company Chimex, octoxyglyceryl behenate (or 2-ethylhexyl glyceryl ether behenate), dioctyl adipate, tartrate of branched C₁₂-C₁₃ dialcohols such as the product sold under the name Cosmacol ETI by Enichem.

Nonionic emulsifying agents include those that can be broadly defined as condensation products of long chain alcohols, e.g. C₈-₃₀ alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these emulsifying agents include those wherein S is a glucose moiety, R is a C₈₋₂₀ alkyl group, and n is an integer of from about 1 to about 9. Commercially available examples of this type of emulsifying agents include decyl polyglucoside (available as APG 325 CS from Henkel) and lauryl polyglucoside (available as APG 600 CS and 625 CS from Henkel).

Other useful nonionic emulsifying agents include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100. Other nonionic emulsifying agents are the condensation products of alkylene oxides with fatty alcohols (i.e. alkylene oxide ethers of fatty alcohols). These materials have the general formula R(X)ₙOR′ wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100 and R′ is H or a C₁₀₋₃₀ alkyl group. Still other nonionic emulsifying agents are the condensation products of alkylene oxides with both fatty acids and fatty alcohols [i.e., wherein the polyalkylene oxide portion is esterified on one end with a fatty acid and etherified (i.e. connected via an ether linkage) on the other end with a fatty alcohol]. These materials have the general formula RCO(X)ₙOR′ wherein R and R″ are C₁₀₋₃₀ alkyl groups, X is —OCH₂CH₂ (i.e. derived from ethylene glycol or oxide) or —OCH₂CHCH₃— (derived from propylene glycol or oxide), and n is an integer from about 6 to about 100. Non-limiting examples of these alkylene oxide derived nonionic emulsifying agents include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10

Other nonionic emulsifying agents include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ethers of polyols, alkyl phosphates, polyoxyalkylene fatty ether phosphates, fatty acid amides, acyl lactylates, and mixtures thereof. Non-limiting examples of these emulsifying agents include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof. Further examples of suitable emulsifiers include mixtures of stearyl octanoate and isopropyl myristate, or mixtures of cetyl octanoate and stearyl octanoate, such those available as Dub Liquide 85 IP, or Dub Liquide from Sterineries de Dubois.

Other emulsifying agents useful herein are fatty acid ester blends based on a mixture of sorbitan or sorbitol fatty acid ester and sucrose fatty acid ester, the fatty acid in each instance being preferably C₈-C₂₄, such as C₁-C₂₀. In one embodiment the fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the trade name Arlatone 2121.

Emulsifying agents can also include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973; these four references are incorporated herein by reference in their entirety.

Exemplary cationic emulsifying agents include those disclosed in U.S. Pat. No. 5,151,209, to McCall et al., issued Sep. 29, 1992; U.S. Pat. No. 5,151,210, to Steuri et al., issued Sep. 29, 1992; U.S. Pat. No. 5,120,532, to Wells et al., issued Jun. 9, 1992; U.S. Pat. No. 4,387,090, to Bolich, issued Jun. 7, 1983; U.S. Pat. 3,155,591, Hilfer, issued Nov. 3, 1964; U.S. Pat. No. 3,929,678, to Laughlin et al., issued Dec. 30, 1975; U.S. Pat. No. 3,959,461, to Bailey et al., issued May 25, 1976; McCutcheon's, Detergents & Emulsifiers, (North American edition 1979) M.C. Publishing Co.; and Schwartz, et al., Surface Active Agents, Their Chemistry and Technology, New York: Interscience Publishers, 1949; all of these documents being incorporated herein by reference in their entirety. The cationic surfactants useful herein include cationic ammonium salts such as quaternary ammonium salts, and amino-amides.

Anionic emulsifying agents are described, e.g., in U.S. Pat. No. 3,929,678, to Laughlin el al., issued Dec. 30, 1975, which is incorporated herein by reference. Non-limiting examples of anionic emulsifying agents include the alkoyl isethionates (e.g., C₁₂-C₃₀), alkyl and alkyl ether sulfates and salts thereof, alkyl and alkyl ether phosphates and salts thereof, alkyl methyl taurates (e.g., C₁₂-C₃₀), and soaps (e.g., alkali metal salts, e.g., sodium or potassium salts) of fatty acids.

Examples of amphoteric and zwitterionic emulsifying agents are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilizing group, e.g., carboxy, sulfonate, sulfate, phosphate, or phosphonate. Examples are alkyl imino acetates, and iminodialkanoates and aminoalkanoates, imidazolinium and ammonium derivatives. Other suitable amphoteric and zwitterionic emulsifying agents are those selected from the group consisting of betaines, sultaines, hydroxysultaines, alkyl sarcosinates (e.g., C₁₂-C₃₀), and alkanoyl sarcosinates.

These silicone emulsifying agents are typically organically modified organopolysiloxanes, also known to those skilled in the art as silicone surfactants. Useful silicone emulsifying agents include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C₂-C₃₀ pendant side chains. Still other useful dimethicone copolyols include materials having various cationic, anionic, amphoteric, and zwitterionic pendant moieties.

Non-limiting examples of dimethicone copolyols and other silicone emulsifying agents useful herein include polydimethylsiloxane polyether copolymers with pendant polyethylene oxide side chains, polydimethylsiloxane polyether copolymers with pendant polypropylene oxide side chains, polydimethylsiloxane polyether copolymers with pendant mixed polyethylene oxide and polypropylene oxide side chains, polydimethylsiloxane polyether copolymers with pendant mixed poly(ethylene)(propylene)oxide side chains, polydimethylsiloxane polyether copolymers with pendant organobetaine side chains, polydimethylsiloxane polyether copolymers with pendant carboxylate side chains, polydimethylsiloxane polyether copolymers with pendant quaternary ammonium side chains; and also further modifications of the preceding copolymers containing pendant C2-C30 straight, branched, or cyclic alkyl moieties. Examples of commercially available dimethicone copolyols useful herein sold by Dow Corning Corporation are Dow Coming® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the trade name ABIL(® WE-09 (available from Goldschmidt). Cetyl dimethicone copolyol is also commercially available as a mixture with hexyl laurate (and) polyglyceryl-3 oleate (and) cetyl dimethicone and is sold under the trade name ABIL® WS-08 (also available from Goldschmidt). Other non-limiting examples of dimethicone copolyols also include lauryl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol Phosphate, and dimethicone copolyol stearate. See International Cosmetic Ingredient Dictionary, Fifth Edition, 1993, which is incorporated by reference herein in its entirety.

In one embodiment, the emulsifying agent is PEG-40 hydrogenated castor oil, such as the commercially available Cremophor® RH 40. In one embodiment, the emulsifying agent is present from between about 0.1 to about 25 wt. %, for example, about 2.5 to about 20 wt. %, such as about 5 to about 15 wt. %, relative to the total weight of the composition.

3. Other Components

The topical compositions of the present invention may include a wide variety of optional components, provided that such optional components are physically and chemically compatible with the essential components described herein, and do not unduly impair stability, efficacy or other use benefits associated with the compositions of the present invention. Optional components may be dispersed, dissolved or the like in the cream base of the present compositions.

As with the above described components, all of the ingredients used in the components according to the embodiments of the present invention intended for application to humans or other animals, e.g., mammals, such as dogs, cats, horses, cows and other domesticated animals, should be non-toxic (or used in non-toxic quantities) and pharmacologically and pharmaceutically safe for such intended use.

Optional components include, for example, aesthetic agents, absorbents (including oil absorbents such as clays an polymeric absorbents), abrasives, anti-caking agents, antifoaming agents, antimicrobial agents (e.g., a compound capable of destroying microbes, preventing the development of microbes or preventing the pathogenic action of microbes and useful, for example, in controlling acne and/or preserving the topical composition), binders, biological additives, buffering agents, bulking agents, chemical additives, cosmetic biocides, denaturants, cosmetic astringents, drug astringents, external analgesics, film formers, humectants, emollients, opacifying agents, fragrances, perfumes, pigments, colorings, essential oils, emollients, skin soothing agents, skin healing agents, pH adjusters, plasticizers, preservatives, preservative enhancers, propellants, reducing agents, skin-conditioning agents, skin protectants, solvents, suspending agents, thickening agents, solubilizing agents, polymers for aiding the film-forming properties and substantively of the composition (such as a copolymer of eicosene and vinyl pyrrolidone, an example of which is available from GAF Chemical Corporation as Ganex) V-220), waxes, sunscreens, sunblocks, ultraviolet light absorbers or scattering agents, antioxidants and/or radical scavengers, chelating agents, sequestrants, anti-acne agents, anti-inflammatory agents, anti-androgens, depilation agents, desquamation agents/exfoliants, organic hydroxy acids, vitamins and derivatives thereof (including water dispersible or soluble vitamins such as Vitamin C and ascorbyl phosphates), compounds which stimulate collagen production, and natural extracts. Such other materials are known in the art. Nonexclusive examples of such materials are described in Harry's Cosmeticology, 7th Ed., Harry & Wilkinson (Hill Publishers, London 1982); in Pharmaceutical Dosage Forms—Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology, 1st Ed. Knowlton & Pearce (Elsevier 1993), can also be used in the present invention.

In one embodiment, the carrier includes an additional thickening agent. For example, mention may be made of other cellulosic ethers, polymeric thickening agents, e.g., acrylic acid polymers, Carbopol® thickeners, etc., xanthan gum, guar gum, and the like, as well as inorganic thickeners/gelling agents. The amount of the thickening agent can be selected to provide the desired product consistency or viscosity to allow for easy application but which will not be too watery or loose so that it will stay where applied. Amounts of thickening agent, up to about 5 wt. % of the total composition, such as between 0.5 and 5 wt. %, such as 2 to 5 wt. %, may be used in the compositions of the present invention.

4. Application

In one embodiment, the amount of formulation to be applied is in the range of from about 0.1 to about 1 milliliter (ml), such as from about 0.1 to 0.5 ml, or from about 0.2 to 0.3 ml. For these embodiments, the application rates the active agents will be contained in the formulation in the amounts as described above, namely, from about 0.001 to 5.0 wt. %, such as 0.05 to 1.5 wt. %, and in another embodiment from about 0.05 to 1.0 wt %.

For a typical, representative formulation according to the invention containing 1 wt. % of PGE-1 and 10 wt. % of 2-n-nonyl-1,3-dioxolane, suitable dosage amounts, as a function of the intended use and area of application, may be from about 0.1 to about 0.5 ml, such as from about 0.2 to 0.3 ml, such as, for example, 0.25 ml.

In one embodiment of the present invention, a method is provided for administering an active agent to the skin of person in need thereof, by applying the compositions of the present invention to the skin of the person in need of treatment.

The compositions of the present invention may be applied to the skin by any suitable means. For example, the composition may be applied directly to the desired area as a cream, or the cream may be applied by means of an aerosol, spray, pump-pack, brush, swab, or other applicator. In one embodiment, the applicator provides either a fixed or variable metered dose application such as a metered dose aerosol, a stored-energy metered dose pump or a manual metered dose pump.

The compositions of the present invention may be propelled by either pump pack or by the use of propellants such as hydrocarbons, hydrofluorocarbons, nitrogen, nitrous oxide, carbon dioxide or ethers, preferably dimethyl ether. The non-occlusive, drug delivery system is preferably in a single phase system as this allows less complicated manufacture and ease of dose uniformity. It may also be necessary to apply a number of dosages on untreated skin to obtain the desired result.

In addition to the above different forms of the composition, the compositions may also be provided for administration by any of the known delivery forms, including, for example, unit dosage and multi-dosage (i.e., multiple unit dosages in a single package or container) forms and bulk forms. As examples of unit dosage forms, mention may be made, for example, of syringes, gelcaps, blister packs, and the like. Bulk forms may be stored in, for example, tubes, bottles, jars, pumps, aerosol containers, and the like, formed of glass, coated metal containers or plastic materials. Again, the formulation and packaging of pharmaceutical products is well within the skill in the art.

The compositions of this invention tend to remain stable against phase separation and product degradation over a wide range of storage conditions. For example, the cream compositions as described above may remain stable over a temperature range of at least from about −20 to 40° C., over periods of several months to years, depending on the storage temperature. It will he understood by one of ordinary skill in the art that stability of a composition to phase separation will be influenced by the conditions under which the composition was formed and stored.

According to an embodiment of the invention, it is possible to use less of the active agent than in current commercial or clinically tested products, thereby lessening the likelihood of adverse reactions, irritation or other side effects.

The compositions of the present invention may also be used to treat diseases in non-human animals. Such veterinary formulations of the present invention can be used with any of the known classes and types of veterinary drug products. For instance, mention may be made of the following: analeptics, such as, for example, diazepam, thiamylal/thipental, midazolam, phentobarbital, phenobarbital; anesthetics, such as, for example, alpha chloralose/chloral hydrate, benzocaine, droperidol/fentanyl, ether, haloethane, isoflurane, ketamine, lidocaine, methohexital, tricaine; antifungals, such as, for example, griseofulvin, ketoconazole; antihistamines, such as, for example, chlorpheniramine, cimetidine, diphenhydramine; antimicrobials, such as, for example, amoxicillin, ampicillin, amoxicillin, clavulinate, cephalosporins, ciproflxacin, clindamycin, doxycycline, enrofloxacin, erthhromycin, gentamicin, lincomycin, minocycline, neomycin, oxytetracycline, penicillin, rifampin, tetracycline, ticarcillin, trimethoprim/sulfonamide; autonomic drugs, such as, for example, atropine, bethanechol, glycopyrrolate; cardiac drugs, such as, for example, captopril, digoxin, epinephrine, furosemide (lasix), procainamide, propanolol, methionine D-L, potassium, selenium/Vitamin E, taurine, Vitamin A & D, Vitamin B complex, Vitamin C, Vitamin D, Vitamin K; gastrointestinal agents, such as, for example, chlorpromazine, cimetidine, metoclopramide, ranitidine; hormones, such as, for example, dexamethasone, dinoprost, estradiol cypionate, fludrocortisone, levothyroxine, methylprednisolone, misoprostol, oxytocin, prednisone/Prednisolone, triamcinolone; muscle relaxants, such as, for example, gallamine, guaifenesin, methocarbinol, metocurine iodide, succinylcholine, tubocurarine; narcotics/analgesics, such as, for example, hydrocodone, naloxone; non-steroidal antiinflammatory/analgesics, such as, for example, acetaminophen, carprofen, flunixin, ibuprofen, ketoprofen, phenylbutazone; respiratory drugs, such as, for example, aminophyline, dextromethorphan, hydrocodone; sedatives, such as, for example, acepromazine, azaperone, diazepam, medetomidine, midazolam, propofol, xylazine; and miscellaneous other drugs, such as, for example, heparin (anticoagulant), insulin (diabetes), methimazole (hyperthyroidism), and the like.

The compositions of the present invention may also include anthelmintic compositions suitable for controlling pathogenic endoparasites encountered in horses in animal keeping and livestock breeding; and in household pets, particularly cats and dogs. They have a favorable toxicity to warm-blooded species. They are effective against all or individual developmental stages of the pests and against resistant and normally sensitive species. The pathogenic endoparasites include Cestodes, Trematodes, Nematodes and Acanthocephala, in particular:

From the order of the Pseudophyllidea, for example: Diphyllobothrium spp., Spirometra spp., Schistocephalus spp.

From the order of the Cyclophyllidea, for example: Mesocestoides spp., Anoplocephala spp., Paranoplocephala spp., Moniezia spp., Taenia spp., Echinococcus spp., Hydatigera spp., Diorchis spp., Dipylidium spp., Joyeuxiella spp., Spyrometra spp.

From the subclass of the Digenea, for example: Schistosoma spp., Fasciola spp., Dicrocoelium spp., Opisthorchis spp.

From the order of the Enoplida, for example: Trichuris spp., Capillaria spp., Trichinella spp.

From the order of the Rhabditia, for example: Micronema spp., Strongyloides spp.

From the order of the Strongylida, for example: Stronylus spp., Triodontophorus spp., Oesophagodontus spp., Trichonema spp., Gyalocephalus spp., Poteriostomum spp., Cyclicocyclus spp., Stephanurus spp., Ancyclostoma spp., Uncinaria spp., Cyathostomum spp., Metastrongylus spp., Dictyocaulus spp., Muellerius spp., Protostrongylus spp., Elaphostrongylus spp., Parelaphostrongylus spp., Crenosoma spp., Paracrenosoma spp., Filaroides spp., Parafilaroides spp., Marshallagia spp., Hyostrongylus spp., Ollulanus spp., Craterostomum spp., Cyclicodontophorus spp., Hyalocephalus spp., Cylindropharynx spp., Caballonema spp., Elaeophorus spp., Dirofilaria spp., Onchocerca spp., Setaria spp.

From the order of the Oxyurida, for example: Oxyuris spp., Enterobius spp.

From the order of the Ascaridia, for example: Ascaris spp., Toxascaris spp., Toxocara spp., Parascaris spp., Probstmangria spp.

From the order of the Spirurida, for example: Thelazia spp., Habronema spp., Draschia spp., Dracunculus spp.

The product can be administered both prophylactically and therapeutically.

Anthelmintics include those which comprise praziquantel or epsiprantel, as hexahydropyrazino derivatives. In addition to the hexahydropyrazinones, the anthelmintic formulations according to the invention can also comprise other active agents. These are phenylguanidines, benzimidazoles or tetrahydropyrimidines. See U.S. Pat. No. 6,025,357 for representative examples, the mention of which is incorporated herein by reference thereto.

The phenylguanidines include, for example, febantel and netobimine.

The benzimidazoles are, for example, febendazole, albendazole, oxibendazole, oxfendazole, mebendazole, flubendazole, parbendazole and luxabendazole.

The tetrahydropyrimidines include, for example, pyrantel, morantel and oxantel.

Other anthelmintic drugs which may be advantageously used in the compositions of this invention include ivermectin, metronidazole, milbemycin oxime, and selamectin.

### EXAMPLES

General Considerations

Preparation of Test Creaytis

Sample emulsion batches of 100 g were prepared in the following manner. The desired quantities of ethanol, Cremophor RH40, and SEPA 0009® (2-n-nonyl-1,3-dioxolane; from MacroChem Corporation, Lexington, Mass.) were combined with slow agitation using a stirrer bar in a jar, and mixed until a clear solution was obtained. To a separate 250 mL beaker was added the desired amount of water. With propeller mixing, the desired amount of Sepigel, Simulgel, or Aristoflex was added to the beaker. Then, the previously prepared mixture of ethanol, Cremophor RH 40 and SEPA 0009® was transferred by pipette to the above described cream base. Finally, Germaben II-E was added to the beaker, and stirring continued for 25 to 30 minutes to obtain the desired cream formulation The composition of the various cream bases are set fourth in Table 1.

PGE₁ (Lot 438020A) was obtained from Spolana (Prague). SEPA 0009® from MacroChem Corporation (Lexington, Mass.). Ethanol was obtained from Aaper Alcohol (Shelbyville, Ky.). Propylene glycol and isopropanol were obtained from Spectrum (New Brunswick, N.J.). Sepigel and Simulgel were obtained from Seppic, Inc. (Fairfield, N.J.). Cremophor RH 40 was supplied by BASF (Wyandotte, N.J.) and Germaben II-E by ISP Sutton (Chatham, N.J.). Water was deionized using a Milli-Q system from Millipore Corp. (Bedford, Mass.).

Physical Results: Test creams were determined to have an acceptable initial physical consistency if they were visually observed to form an emulsion upon mixing that did not readily separate under ambient conditions.

Stability to centrifugation: Test creams were judged to be stable to centrifugation if they were unchanged visibly (naked eye and microscope) after centrifugation at 1300G for up to two hours, if no inhomogeneities were observed (in particular the crystallization of drug), and if assays showed potency within 10% of calculated active agent and SEPAL. Representative products (formulation numbers 11, 13 and 16) showing continued stability (1 week minimum) were submitted for transdermal testing, as further described below.

TABLE 1

| | FORMULATION | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| SEPA 0009 | 5 | 5 | 5 | 5 | 5 | 5 | 5 | 5 | 10 | 5 | 10 | 10 |
| PGE1 | | 1 | 2 | 1 | 2 | 1 | 2 | 1 | 1 | 1 | 2 | 1 |
| Testosterone | 1 | | | | | | | | | | | |
| Ibuprofen | | | | | | | | | | | | |
| Hydroquinone | | | | | | | | | | | | |
| Methylphenidate HCl | | | | | | | | | | | | |
| Sepigel 305 | | 4 | 4 | 4 | 4 | 4 | 4 | 4 | 4 | 4 | 4 | |
| Simulgel 600 | 4 | | | | | | | | | | | 4 |
| Aristoflex | | | | | | | | | | | | |
| Cetearyl Octanoate | 5 | | | 5 | 5 | | | | | | | |
| Cremophor RH 40 | 15 | 5 | 5 | 5 | 5 | 5 | 5 | 5 | 5 | | 5 | 5 |
| Tween 80 | | | | | | | | | | | | |
| Tween 60 | | | | | | | | | | | | |
| Volpo S-20 | | | | | | | | | | | | |
| Cetomacrogol 1000 | | | | | | | | | | | | |
| Glucamate SSE-20 | | | | | | | | | | | | |
| Phospholipon 90H | | | | | | | | | | | | |
| Sesame Oil | | | | | | | | | | | | |
| Dub Liquide | | | | | | | | | | | | |
| White Mineral Oil | | | | | | | | | | | | |
| Ethanol | 25 | 40 | 40 | 40 | 40 | 40 | 40 | 50 | 40 | 50 | 40 | 40 |
| Miglyol 812N (Caprylic/ | 5 | | | | | | | | | | | |
| capric triglyceride) | | | | | | | | | | | | |
| Propylene Glycol | 5 | | | | | | | | | | | |
| Germaben II-E | 1 | 1 | 1 | 1 | 1 | 1 | 1 | 1 | 1 | 1 | 1 | 1 |
| H2O | 34 | 43.5 | 42.5 | 39 | 38 | 44 | 43 | 34 | 39 | 39 | 38 | 39 |
| 1% Citric Acid | | 0.5 | 0.5 | | | | | | | | | |
| Ascorbic acid | | | | | | | | | | | | |
| NaOH (2.5%) | | | | | | | | | | | | |
| Total | 100 | 100 | 100 | 100 | 100 | 100 | 100 | 100 | 100 | 100 | 100 | 100 |
| pH | 6.9 | 5.1 | 4.9 | 5.3 | 5.1 | 5.3 | 5.0 | 5.6 | 5.4 | 5.2 | 5.2 | 5.6 |
| Physical Results | P | P | P | P | P | P | P | P | P | P | P | P |
| Stable to centrifugation? | nt | Y | Y | Y | Y | Y | Y | Y | Y | Y | Y | Y |
| | FORMULATION | | | | | | | | | | | |
| | 13 | 14 | 15 | 16 | 17 | 18 | 19 | 20 | 21 | 22 | 23 | 24 |
| SEPA 0009 | 10 | 10 | 7.5 | 7.5 | 5 | 7.5 | 7.5 | 7.5 | 7.5 | 7.5 | 5 | 7.5 |
| PGE1 | 1 | 2 | 2 | 2 | 1 | 1 | 1 | 1.5 | 1.5 | 1.5 | | |
| Testosterone | | | | | | | | | | | 1 | 1 |
| Ibuprofen | | | | | | | | | | | | |
| Hydroquinone | | | | | | | | | | | | |
| Methylphenidate HCl | | | | | | | | | | | | |
| Sepigel 305 | | | 4 | | | | 4 | 4 | | 4 | | |
| Simulgel 600 | 4 | 4 | | 4 | 4 | 4 | | | 4 | | 5 | 5 |
| Aristoflex | | — | | | | | | | | | | |
| Cetearyl Octanoate | | | | | | | | | | | | |
| Cremophor RH 40 | 2.5 | 2.5 | 2.5 | 2.5 | 2.5 | 2.5 | 2.5 | 2.5 | 2.5 | 2.5 | 2.5 | 2.5 |
| Tween 80 | | | | | | | | | | | | |
| Tween 60 | | | | | | | | | | | | |
| Volpo S-20 | | | | | | | | | | | | |
| Cetomacrogol 1000 | | | | | | | | | | | | |
| Glucamate SSE-20 | | | | | | | | | | | | |
| Phospholipon 90H | | | | | | | | | | | | |
| Sesame Oil | | | | | | | | | | | | |
| Dub Liquide | | | | | | | | | | | | |
| White Mineral Oil | | | | | | | | | | | | |
| Ethanol | 40 | 40 | 40 | 40 | 40 | 40 | 40 | 40 | 40 | 40 | 40 | 40 |
| Miglyol 812N (Caprylic/ | | | | | | | | | | | | |
| capric triglyceride) | | | | | | | | | | | | |
| Propylene Glycol | | | | | | | | | | | 5 | 5 |
| Germaben II-E | 1 | 1 | 1 | 1 | 1 | 1 | 1 | 1 | 1 | 1 | 1 | 1 |
| H2O | 41.5 | 40.5 | 43 | 43 | 46.5 | 44 | 44 | 44 | 44 | 44 | 41 | 38 |
| 1% Citric Acid | | | | | | | | | | | | |
| Ascorbic acid | | | | | | | | | | | | |
| NaOH (2.5%) | | | | | | | | | | | | |
| Total | 100 | 100 | 100 | 100 | 100 | 100 | 100 | 100 | 100 | 100 | 100 | 100 |
| pH | 5.6 | 5.3 | 4.9 | 5.1 | 5.3 | 5.3 | 5.1 | 4.9 | 5.2 | 5.2 | 6.8 | 6.6 |
| Physical Results | P | P | P | P | P | P | P | P | P | P | P | P |
| Stable to | Y | Y | Y | Y | Y | Y | Y | Y | Y | Y | Y | Y |
| centrifugation? | | | | | | | | | | | | |
| | FORMULATION | | | | | | | | | | | |
| | 25 | 26 | 27 | 28 | 29 | 30 | 31 | 32 | 33 | 34 | 35 | 36 |
| SEPA 0009 | 7.5 | 10 | 5 | 5 | 5 | 5 | 5 | 5 | 5 | 10 | 5 | 5 |
| PGE1 | | | | | | | | | | | | |
| Testosterone | 1 | 1 | 1 | 1 | 1 | 1 | 1 | 1 | 1 | 1 | 1 | 1 |
| Ibuprofen | | | | | | | | | | | | |
| Hydroquinone | | | | | | | | | | | | |
| Methylphenidate HCl | | | | | | | | | | | | |
| Sepigel 305 | | | | | | | | | | | | |
| Simulgel 600 | 5 | 5 | 5 | 5 | 5 | 5 | 5 | 5 | 5 | 5 | | |
| Aristoflex | | | | | | | | | | | 0.5 | 1 |
| Cetearyl Octanoate | | | | | | | | | | | | |
| Cremophor RH 40 | 5 | 2.5 | 2.5 | | | | | | | 2.5 | 2.5 | 2.5 |
| Tween 80 | | | | 2.5 | | | | | | | | |
| Tween 60 | | | | | 2.5 | | | | | | | |
| Volpo S-20 | | | | | | 2.5 | | | | | | |
| Cetomacrogol 1000 | | | | | | | 2.5 | | | | | |
| Glucamate SSE-20 | | | | | | | | 2.5 | | | | |
| Phospholipon 90H | | | | | | | | | 2.5 | | | |
| Sesame Oil | | | | | | | | | | | | |
| Dub Liquide | | | | | | | | | | | | |
| White Mineral Oil | | | | | | | | | | | | |
| Ethanol | 40 | 40 | 40 | 40 | 40 | 40 | 40 | 40 | 40 | 40 | 40 | 40 |
| Miglyol 812N (Caprylic/ | | | | | | | | | | | | |
| capric triglyceride) | | | | | | | | | | | | |
| Propylene Glycol | 5 | 5 | 5 | 5 | 5 | 5 | 5 | 5 | 5 | 5 | 5 | 5 |
| Germaben II-E | 1 | 1 | 1 | 1 | 1 | 1 | 1 | 1 | 1 | 1 | 1 | 1 |
| H2O | 36 | 36 | 41 | 41 | 41 | 41 | 41 | 41 | 41 | 36 | 45 | 45 |
| 1% Citric Acid | | | | | | | | | | | | |
| Ascorbic acid | | | | | | | | | | | | |
| NaOH (2.5%) | | | | | | | | | | | | |
| Total | 100 | 100 | 100 | 100 | 100 | 100 | 100 | 100 | 100 | 100 | 100 | 100 |
| pH | 6.6 | 6.5 | 6.7 | 4.9 | 6.4 | 6.3 | 6.4 | 6.5 | 6.5 | 6.7 | 5.3 | 6.2 |
| Physical Results | P | P | P | P | P | P | P | P | P | P | P | P |
| Stable to centrifugation? | Y | Y | Y | N | N | N | N | N | N | Y | nt | Y |

| | FORMULATION | | | | | | | | | | | |
|---|---|---|---|---|---|---|---|---|---|---|---|---|
| | 37 | 38 | 39 | 40 | 41 | 42 | 43 | 44 | 45 | 46 | 47 | 48 |
| SEPA 0009 | 5 | 0 | 0 | 5 | 10 | 10 | 10 | 7.5 | 5 | 5 | 5 | 5 |
| PGE1 | | | | | 1 | 1 | 1 | | | | | |
| Testosterone | 1 | | | | | | | | | | | |
| Ibuprofen | | | | | | | | | 5 | 5 | 5 | 5 |
| Hydroquinone | | 3 | 3 | 3 | | | | | | | | |
| Methylphenidate | | | | | | | | 2 | | | | |
| HCI | | | | | | | | | | | | |
| Sepigel 305 | | | | | | | | | | | | |
| Simulgel 600 | | 3 | 3.5 | 3.5 | 4 | 4.5 | 5 | 4 | 5 | 5 | 5 | 2.5 |
| Aristoflex | 1 | | | | | | | | | | | |
| Cetearyl Octanoate | | | | | | | | | | | | |
| Cremophor RH 40 | | | | | 2.5 | 2.5 | 2.5 | 1 | 2.5 | 2.5 | 1 | 2.5 |
| Tween 80 | | | | | | | | | | | | |
| Tween 60 | | | | | | | | | | | | |
| Volpo S-20 | | | | | | | | | | | | |
| Cetomacrogol 1000 | | | | | | | | | | | | |
| Glucamate SSE-20 | | | | | | | | | | | | |
| Phospholipon 90H | | | | | | | | | | | | |
| Sesame Oil | | | | | | | | | | | | |
| Dub Liquide | | | | | | | | | | | | |
| White Mineral Oil | | | | | | | | | | | | |
| Ethanol | 40 | | | | 41 | 45 | 45 | 40 | 40 | 30 | 40 | 40 |
| Miglyol 812N | | | | | | | | | | | | |
| (Caprylic/capric | | | | | | | | | | | | |
| triglyceride) | | | | | | | | | | | | |
| Propylene Glycol | 5 | | | | | | | | | 5 | 5 | 5 |
| Germaben II-E | 1 | | | | | | | | 1 | 1 | 1 | 1 |
| H2O | 47 | 89 | 88 | 83 | 41.5 | 37 | 36.5 | 54.5 | 41.5 | 46.5 | 38 | 39 |
| 1% Citric Acid | | | | | | | | | | | | |
| Ascorbic acid | | 0.5 | 0.5 | 0.5 | | | | | | | | |
| NaOH (2.5%) | | 5 | 5 | 5 | | | | | Y | Y | Y | Y |
| Total | 100 | 100 | 100 | 100 | 100 | 100 | 100 | 100 | 100 | 100 | 100 | 100 |
| pH | 6.2 | — | 5.2 | 5.2 | 5.5 | 4.8 | 4.8 | nt | 7.02 | 6.62 | 6.61 | 6.62 |
| Physical Results | P | F | F | F | P | P | P | F | F | F | F | F |
| Stable to | N | nt | nt | nt | Y | Y | Y | nt | nt | nt | nt | nt |
| centrifugation? | | | | | | | | | | | | |

| | FORMULATION | | | | | | | | | | | |
|---|---|---|---|---|---|---|---|---|---|---|---|---|
| | 49 | 50 | 51 | 52 | 53 | 54 | 55 | 56 | 57 | 58 | 59 | 60 |
| SEPA 0009 | 5 | 5 | 5 | 5 | 5 | 5 | 5 | 5 | 5 | 5 | 5 | 5 |
| PGE1 | | | | | | | | | | | | |
| Testosterone | | | | | | | | | | | | |
| Ibuprofen | 5 | 5 | 5 | 5 | 5 | 5 | 5 | 5 | 5 | 5 | 5 | 5 |
| Hydroquinone | | | | | | | | | | | | |
| Methylphenidate | | | | | | | | | | | | |
| HCl | | | | | | | | | | | | |
| Sepigel 305 | | | | | | | | | | | | |
| Simulgel 600 | 5 | 5 | 5 | 5 | 5 | 5 | 5 | 5 | 2.5 | 5 | 5 | 5 |
| Aristoflex | | | | | | | | | | | | |
| Cetearyl | | | | | | | | | | | | |
| Octanoate | | | | | | | | | | | | |
| Cremophor RH 40 | 2.5 | 2.5 | 5 | 7 | 2.5 | 2.5 | 5 | 7 | 2.5 | 1 | 0 | 2.5 |
| Tween 80 | | | | | | | | | | | | |
| Tween 60 | | | | | | | | | | | | |
| Volpo S-20 | | | | | | | | | | | | |
| Cetomacrogol | | | | | | | | | | | | |
| 1000 | | | | | | | | | | | | |
| Glucamate SSE-20 | | | | | | | | | | | | |
| Phospholipon 90H | | | | | | | | | | | | |
| Sesame Oil | | | | | | | | | | | | |
| Dub Liquide | | | | | | | | | | | | |
| White Mineral Oil | | | | | | | | | | | | |
| Ethanol | 30 | 20 | 40 | 40 | 15 | 15 | 15 | 15 | 15 | 15 | 15 | 10 |
| Miglyol 812N | | | | | | | | | | | | |
| (Caprylic/ | | | | | | | | | | | | |
| capric triglyceride) | | | | | | | | | | | | |
| Propylene Glycol | | | 5 | 5 | | 5 | | | | | | |
| Germaben II-E | 1 | 1 | 1 | 1 | 1 | 1 | 1 | 1 | 1 | 1 | 1 | 1 |
| H2O | 51.5 | 61.5 | 34 | 32 | 66.5 | 61.5 | 64 | 62 | 69 | 68 | 69 | 71.5 |
| 1% Citric Acid | | | | | | | | | | | | |
| Ascorbic acid | | | | | | | | | | | | |
| NaOH (2.5%) | Y | Y | Y | Y | Y | Y | Y | Y | Y | Y | Y | Y |
| Total | 100 | 100 | 100 | 100 | 100 | 100 | 100 | 100 | 100 | 100 | 100 | 100 |
| pH | 6.62 | 6.65 | 6.63 | 6.54 | 6.64 | 6.64 | 6.63 | 6.65 | 6.63 | 6.64 | 6.65 | 6.63 |
| Physical Results | F | P | F | F | P | P | P | P | P | P | P | P |
| Stable to | nt | Y | nt | nt | Y | N | Y | Y | N | Y | N | Y |
| centrifugation? | | | | | | | | | | | | |

| | FORMULATION | | | | | | | | | |
|---|---|---|---|---|---|---|---|---|---|---|
| | 61 | 62 | 63 | 64 | 65 | 66 | 67 | 68 | 69 | 70 |
| SEPA 0009 | 0 | 0 | 0 | 0 | 0 | 0 | 0 | 0 | 0 | 5 |
| PGE1 | | | | | | | | | | |
| Testosterone | | | | | | | | | | |
| Ibuprofen | 5 | 5 | 5 | 5 | 5 | 5 | 5 | 5 | 5 | 5 |
| Hydroquinone | | | | | | | | | | |
| Methylphenidate HCl | | | | | | | | | | |
| Sepigel 305 | | | | | | | | | | |
| Simulgel 600 | 5 | 5 | 5 | 5 | 5 | 5 | 5 | 5 | 5 | 5 |
| Aristoflex | | | | | | | | | | |
| Cetearyl Octanoate | | | | | | | | | | |
| Cremophor RH 40 | 2.5 | 2.5 | 5 | 7 | 1 | 2.5 | 2.5 | 2.5 | 2.5 | 7 |
| Tween 80 | | | | | | | | | | |
| Tween 60 | | | | | | | | | | |
| Volpo S-20 | | | | | | | | | | |
| Cetomacrogol 1000 | | | | | | | | | | |
| Glucamate SSE-20 | | | | | | | | | | |
| Phospholipon 90H | | | | | | | | | | |
| Sesame Oil | | | | | | | | | | |
| Dub Liquide | | | | | | | | | | |
| White Mineral Oil | | | | | | | | 5 | 5 | |
| Ethanol | 20 | 15 | 15 | 15 | 15 | 17.5 | 18.5 | 20 | 15 | 20 |
| Miglyol 812N (Caprylic/ | | | | | | | | | | |
| capric triglyceride) | | | | | | | | | | |
| Propylene Glycol | | | | | | | | | | |
| Germaben II-E | 1 | 1 | 1 | 1 | 1 | 1 | 1 | 1 | 1 | 1 |
| H2O | 66.5 | 71.5 | 69 | 67 | 73 | 69 | 68 | 61.5 | 66.5 | 57 |
| 1% Citric Acid | | | | | | | | | | |
| Ascorbic acid | | | | | | | | | | |
| NaOH (2.5%) | Y | Y | Y | Y | Y | Y | Y | Y | Y | Y |
| Total | 100 | 100 | 100 | 100 | 100 | 100 | 100 | 100 | 100 | 100 |
| pH | 6.63 | 6.65 | 6.64 | 6.65 | 6.64 | 6.66 | 6.65 | 6.63 | 6.66 | 6.75 |
| Physical Results | P | P | P | P | P | F | P | P | F | P |
| Stable to centrifugation? | Y | N | N | Y | N | nt | Y | N | nt | N |

FORMULATION

| | 71 | 72 | 73 | 74 |
|---|---|---|---|---|
| SEPA 0009 | 0 | 0 | 0 | 0 |
| PGE1 | | | | |
| Testosterone | | | | |
| Ibuprofen | 5 | 5 | 5 | 5 |
| Hydroquinone | | | | |
| Methylphenidate HCl | | | | |
| Sepigel 305 | | | | |
| Simulgel 600 | 5 | 5 | 5 | 5 |
| Aristoflex | | | | |
| Cetearyl Octanoate | | | | |
| Cremophor RH 40 | 2.5 | 2.5 | 2.5 | 2.5 |
| Tween 80 | | | | |
| Tween 60 | | | | |
| Volpo S-20 | | | | |
| Cetomacrogol 1000 | | | | |
| Glucamate SSE-20 | | | | |
| Phospholipon 90H | | | | |
| Sesame Oil | | | | 5 |
| Dub Liquide | 5 | 5 | 5 | |
| White Mineral Oil | | | | |
| Ethanol | 15 | 20 | 30 | 20 |
| Miglyol 812N (Caprylic/ | | | | |
| capric triglyceride) | | | | |
| Propylene Glycol | | | | |
| Germaben II-E | 1 | 1 | 1 | 1 |
| H2O | 66.5 | 61.5 | 51.5 | 61.5 |
| 1% Citric Acid | | | | |
| Ascorbic acid | | | | |
| NaOH (2.5%) | Y | Y | Y | Y |
| Total | 100 | 100 | 100 | 100 |
| pH | 6.64 | 6.65 | 6.64 | 6.64 |
| Physical Results | P | P | P | P |
| Stable to centrifugation? | Y | N | N | Y |

P = Pass; F = Fail; Y = Yes; N= No; nt = not tested

Transdermal Test Procedures

Preparation of skin: Human cadaver skin was obtained from AATB accredited tissue banks. The tissue was recovered within 15 hours of death or within 24 hours if the body was refrigerated. It was retrieved under aseptic technique, quarantined, and placed in antibiotics (Penicillin 50,000 U/Gentamicin 10 mg). Prior to cryopreservation, dermatomed skin grafts were rinsed, cut and measured. Skin thickness ranged between 250-800 μm. Skin grafts were then placed between gauze, folded and placed into a cryo-foil package with 5 mL of cryo-protectant (15% (w/w) glycerin in Lactated Ringer, USP). Antibiotics were added to the cryoprotectant. The packages were sealed and the tissue was frozen using control-rate freezing (1° C./5 minutes) to a temperature of −70° C. or lower. Skin samples were stored in a freezer at −20° C. One day prior to use, the skin was quickly thawed at 4° C., rinsed with tap water, washed with phosphate buffered saline solution, and blotted dry. Skin circlets were punched and mounted onto diffusion cells. All donors from whom these allografts were derived had been tested and found negative for hepatitis B surface antigen and antibodies human immunodeficiency viruses (HIV-1 and HIV-2), hepatitis C virus (HCV), hepatitis B surface antigen (HbsAg), hepatitis B core (HbcAB Igm), HTLV-1, and syphilis by FDA approved tests in CLIA approved laboratories.

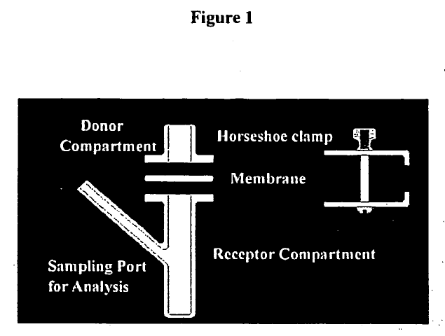

Skin permeation methodology: Percutaneous absorption was measured using horizontal glass diffusion cells consisting of a donor and a receptor compartment (Cf. FIG. 1). Such cells are referred to as Franz-type diffusion cells, or static cells, and were supplied by Crown Glass Company (Somerville, N.J., U.S.A). Dermatomed skin samples were punched out with a metallic punch, and placed between the two halves of a diffusion cell, the stratum corneum facing the donor compartment. The test apparatus is shown schematically in FIG. 1. The area available for diffusion was 0.635 cm² and the receptor compartment volume was 5.5 mL. The receptor chamber was filled, so the liquid interfaced with the skin membrane, with approximately 5 mL phosphate buffered saline (pH 7.4) and allowed to equilibrate to the correct temperature. Temperature of the skin surface was maintained at 32° C. throughout the experiment by placing diffusion cells into dry block heater set on 37° C. The receptor compartment contents were continuously agitated by small PTFE-coated magnetic stirring bars at 600 rpm.

Skin samples were allowed to equilibrate with phosphate buffered saline with 0.2% Volpo 20 for at least one hour before application of test formulations on the morning of the experiment. Formulations were applied using a 50 μL dispensing pipette (VWR). The pipette was weighed before and after application and the exact amounts applied were recorded. Target application amount was between 5 and 10 mg/cm² of formulation. Following application of the products, the entire receptor phase was removed at regular time intervals, 2, 4, 6, 8, and 24 hours, using a 5 mL syringe. The receptor compartment was then refilled with fresh temperature equilibrated receptor medium. Volumes collected were measured and recorded. Each test formulation was tested simultaneously in a minimum of 6 diffusion cells.

Following the final receptor phase sample, the residual drug remaining on the surface of the skin was determined.

Receptor sample preparation: The analytical method used to measure PGE-1 concentration was an indirect method in which PGE-1 concentration in samples was determined after derivatization of PGE-1 into PGB (PGB being a more stable molecule than PGE-1). The derivatization process consisted of treating a 1 mL sample with 1 mL of 1N NaOH for 2 hours at 50° C. After 2 hours, the reaction was stopped by adding 100 μL of HPLC grade 85% o-phosphoric acid to each sample. Receptor fluid samples were then placed into 2 mL HPLC vials and analyzed for PGB content.

Analytical methodology: Analytical determinations were made by high performance liquid chromatography (HPLC) using an Agilent 1050 LC module equipped with a variable wavelength detector, column oven, in-line degasser and autosampler. Details regarding HPLC conditions used for the PGB are described in a separate report. In all cases, the amount of PGB present in the samples was determined by measuring the PGB peak against a 6-point minimum calibration curve.

Statistical methodology: The data were expressed as flux (μg/cm²/hour) units, cumulative amount absorbed (μg/cm²) and percentage of dose applied versus time. All data represent the average of the n=6 replicates for each experiment and associated standard error. Group means were evaluated for statistical significance by two-tailed, paired t-tests based on equal or unequal variance assumption as appropriate using Excel® statistical package (Microsoft Inc.). No adjustment was made to the results for this report for multiple comparisons.

The data obtained from the above measurements is reported graphically in the figures.

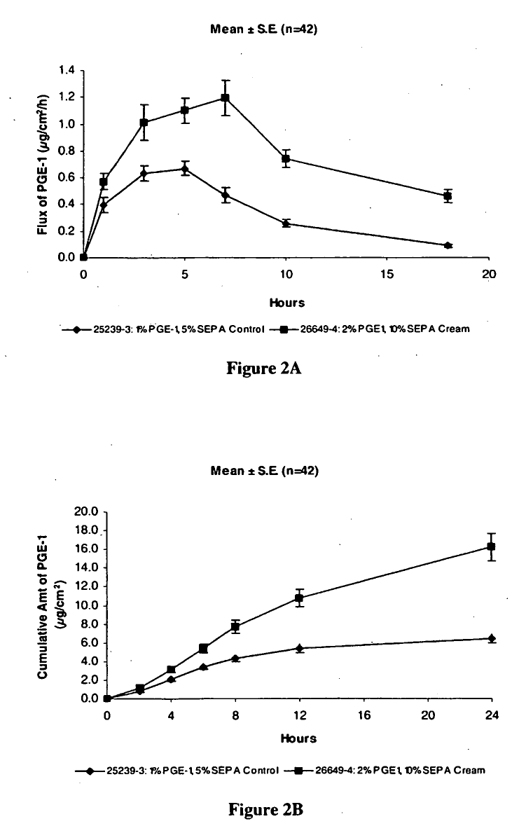

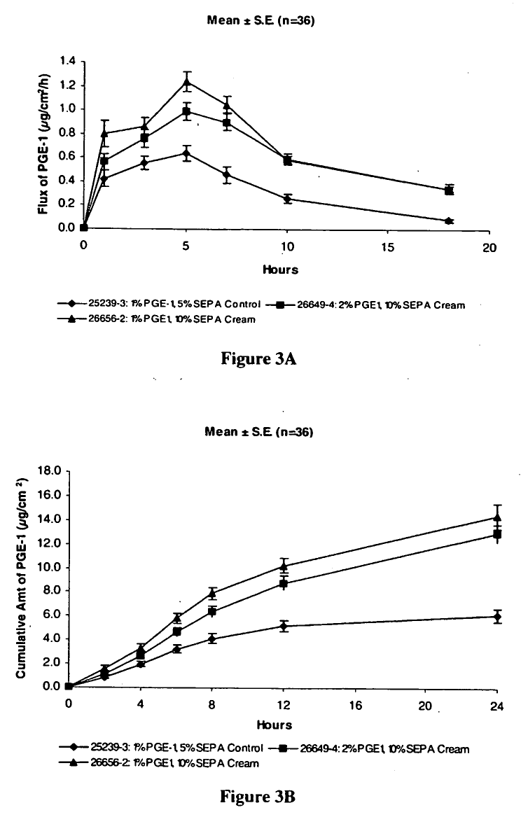

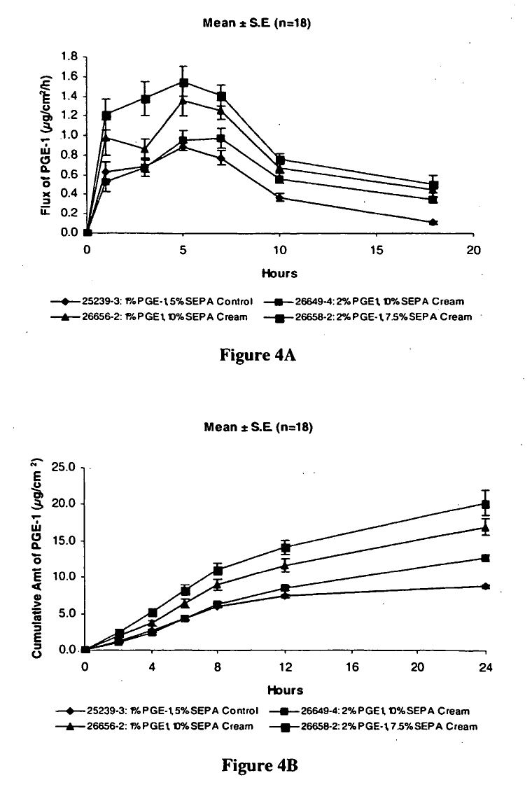

FIGS. 2A and 2B show graphs illustrating average flux (FIG. 2A) and cumulative delivery (FIG. 2B) of PGE, across human skin vs. time for formulation number 11 (identified as “Sample 26649-4”) in Table 1, against a clear gel control of 1% PGE, 5% SEPA-0009, 1% Klucel HP, 65.1% ethanol, and 27.9% water. FIGS. 3A and 3B show graphs illustrating average flux (FIG. 3A) and cumulative delivery (FIG. 3B) of PGE₁ across human skin vs. time for formulations 11 (identified as “Sample 26649-4”) and 13 (identified as “Sample 26656-2”) in Table 1, against a clear gel control of 1% PGE1, 5% SEPA-0009, 1% Klucel HF, 65.1% ethanol, and 27.9% water. FIGS. 4A and 4B show graphs illustrating average flux (FIG. 4A) and cumulative delivery (FIG. 4B) of PGE₁ across human skin vs. time for formulations 11 (identified as “Sample 26649-4”), 13 (identified as “Sample 26656-2”) and 16 (identified as “Sample 26658-2”) in Table 1, against a clear gel control of 1% PGE 1, 5%. SEPA-0009, 1% Klucel HF, 65.1% ethanol, and 27.9% water.

Having described specific embodiments of the present invention, it will be understood that many modifications thereof will readily appear or may be suggested to those skilled in the art, and it is intended therefore that this invention is limited only by the spirit and scope of the following claims.

1. A vanishing cream base composition suitable for topical application of an active agent to an animal or plant comprising:
a. from about 20 to about 70 wt. % water, based on the total weight of cream base composition;
b. at least about 10 wt. %, based on the total weight of cream base, of at least one alcohol;
c. at least one polymeric thickening agent comprising a nitrogen containing acrylic unit;
d. a penetration enhancing effective amount of a skin penetration enhancing compound; and
e. an emulsifying agent;
wherein said cream base is stable upon storage at ambient conditions, and clarifies at a temperature greater than 30° C.

2. A vanishing cream composition suitable for topical application of an active agent to an animal or plant comprising:
a. from about 20 to about 70 wt. % water, based on the total weight of cream base composition;
b. at least about 10 wt. %, based on the total weight of cream base, of at least one alcohol;
c. at least one polymeric thickening agent comprising a nitrogen containing acrylic unit;
d. a penetration enhancing effective amount of a skin penetration enhancing compound;
e. an emulsifying agent; and
f. an active agent;
wherein said cream base is stable upon storage at ambient conditions, and clarifies at a temperature greater than 30° C.

3. The composition of claim 2 wherein said emulsifying agent is polyoxyl 40 hydrogenated castor oil.

4. The composition of claim 3 wherein said polyoxyl 40 hydrogenated castor oil is present in an amount from about 2 to about 17 wt. % based on total weight of the cream base composition.

5. The composition of claim 2 having less than about 45 wt. % water.

6. The composition of claim 2 having less than about 40 wt. % water.

7. The composition of claim 2 having less than about 35 wt. % water.

8. The composition of claim 2 wherein said at least one alcohol is present in at least about 35 wt. %.

9. The composition of claim 2 wherein said at least one alcohol is present in at least about 40 wt. %.

10. The composition of claim 2 wherein said at least one alcohol is present in at least about 45 wt. %.

11. The composition of claim 2 wherein said at least one alcohol comprises ethanol.

12. The composition of claim 2 wherein said polymeric thickening agent comprising a nitrogen containing acrylic unit is present in an amount from about 0.2 to about 10 wt. %, relative to the total weight of the cream base composition.

13. The composition of claim 2 wherein said polymeric thickening agent comprising a nitrogen containing acrylic unit is present in an amount from about 3 to about 7 wt. %, relative to the total weight of the cream base composition.

14. The composition of claim 2 wherein said polymeric thickening agent comprising a nitrogen containing acrylic unit is present in an amount from about 4 to about 6 wt. %, relative to the total weight of the cream base composition.

15. The composition of claim 2 further comprising Germaben II-E.

16. The composition of claim 2 wherein said skin penetration enhancing compound is a 1,3-dioxane, 1,3-dioxolane or acetal substituted with a C₆ to C₁₂-hydrocaryl group.

17. The composition of claim 16 wherein said skin penetration enhancing compound is 2-n-nonyl-1,3-dioxolane.

18. The composition of claim 17 wherein said 2-n-nonyl-1,3-dioxolane is present in an amount from about 5 to about 20 wt. %, relative to the total weight of the cream base composition.

19. The composition of claim 17 wherein said 2-n-nonyl-1,3-dioxolane is present in an amount from about 7 to about 15 wt. %, relative to the total weight of the cream base composition.

20. The composition of claim 2 wherein said thickener is an ammonium acryloyldimethlaurate-beheneth-25 methacrylate crosspolymer.

21-45. (canceled)
